SEQUENCE LISTING (124 sequences)

SEQ ID NO: 1 - DNA, 21 bp, Artificial Sequence
  Forward primer for the HPC22, HPC37 general microbial 16S RNA core coding core16s-rna gene sequences
tcctacggaggcagcagtnnn

SEQ ID NO: 2 - DNA, 27 bp, Artificial Sequence
  Reverse primer for the HPC22, HPC37 general microbial 16S RNA core coding core16s-rna gene sequences
tattaccgcgnnngctgctggcacnnn

SEQ ID NO: 3 - DNA, 24 bp, Artificial Sequence
  Fluorescent labelled Probe for the HPC22, HPC37 general microbial 16S RNA core coding core16s-rna gene sequences
taccagggtatctaatcctgttnn

SEQ ID NO: 4 - DNA, 20 bp, Artificial Sequence
  Forward primer for the HPC22, HPC37 GAPDH glyceraldehyde phosphate dehydrogenase enzyme coding gapdh gene sequences
gatctgctcgtaagttgnnn

SEQ ID NO: 5 - DNA, 18 bp, Artificial Sequence
  Reverse primer for the HPC22, HPC37 GAPDH glyceraldehyde phosphate dehydrogenase enzyme coding gapdh gene sequences
aaaccgttgatggcccnn

SEQ ID NO: 6 - DNA, 22 bp, Artificial Sequence
  Fluorescent labelled Probe for the HPC22, HPC37 GAPDH glyceraldehyde phosphate dehydrogenase enzyme coding gapdh gene sequences
nnntttagcagcaccggtagnn

SEQ ID NO: 7 - DNA, 26 bp, Artificial Sequence
  Forward primer for the Coliforms GAL beta-galactosidase coding lacZ gene sequences
natgaaagctggctacaggaaggccn

SEQ ID NO: 8 - DNA, 26 bp, Artificial Sequence
  Reverse primer for the Coliforms GAL beta-galactosidase coding lacZ gene sequences
caccatgccgtgggtttcaatattnn

SEQ ID NO: 9 - DNA, 29 bp, Artificial Sequence
  Fluorescent labelled Probe for the Coliforms GAL beta-galactosidase coding lacZ gene sequences
cgtttgccgtctgaatttgacctgagnnn

SEQ ID NO: 10 - DNA, 26 bp, Artificial Sequence
  Forward primer for the Coliforms GUS beta-glucuronidase coding uidA gene sequences
ntggtaattaccgacgaaaacggcnn

SEQ ID NO: 11 - DNA, 27 bp, Artificial Sequence
  Reverse primer for the Coliforms GUS beta-glucuronidase coding uidA gene sequences
gtaatgctctacaccacgccgaacacn

SEQ ID NO: 12 - DNA, 29 bp, Artificial Sequence
  Fluorescent labelled Probe for the Coliforms GUS beta-glucuronidase coding uidA gene sequences
ngtaatgctctacaccacgccgaacacnn

SEQ ID NO: 13 - DNA, 528 bp, Artificial Sequence
  HPC22, HPC37 general microbial 16S RNA core coding core16s-rna gene sequences, the example of Bacillus cereus gi|218158707|gb|CP001176.1| Bacillus cereus B4264, complete
gccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtgctagttgaataagctggcaccttgacggtacctaaccagaaagccacggctaactacgtgccagcagccgcggtaatacgtaggtggcaagcgttatccggaattattgggcgtaaagcgcgcgcaggtggtttcttaagtctgatgtgaaagcccacggctcaaccgtggagggtcattggaaactgggagacttgagtgcagaagaggaaagtggaattccatgtgtagcggtgaaatgcgtagagatatggaggaacaccagtggcgaaggcgactttctggtctgtaactgacactgaggcgcgaaagcgtggggagcaaacaggattagataccctggtagtccacgccgtaaacgatgagtgctaagtgttag

SEQ ID NO: 14 - DNA, 19 bp, Artificial Sequence
  Template to Forward primer
tcctacgggaggcagcagt

SEQ ID NO: 15 - DNA, 22 bp, Artificial Sequence
  Template to Reverse primer
aacaggattagataccctggta

SEQ ID NO: 16 - DNA, 21 bp, Artificial Sequence
  Template to Fluorescent labelled Probe
gtgccagcagccgcggtaata

SEQ ID NO: 17 - DNA, 350 bp, Artificial Sequence
  HPC22, HPC37 GAPDH glyceraldehyde phosphate dehydrogenase enzyme coding gapdh gene sequences, the example of Shigella dysenteriae gi|812395301364349-1365344 |Shigella dysenteriae gdh Sd197
cgttgaaatcggtagatactacgtcatcttcggtgtagcccagaacgcctttcatttcgccttcagcagcagctttaacggcagctttgatctgctcgtaagttgcagctttttccagacgaacggtcaggtcaactacggatacgttcggggtcggaacgcggaacgccataccagtcagtttgccattcagttctggcagtactttacctacagctttagcagcaccggtagaggacgggatgatgttctgggaagcgccgcggccgccgcgccagtctttgtgagacgggccatcaacggttttctgagtagcggtagtagcgtgaacggtggtcatcagaccttcg

SEQ ID NO: 18 - DNA, 17 bp, Artificial Sequence
  Template to Forward primer
gatctgctcgtaagttg

SEQ ID NO: 19 - DNA, 16 bp, Artificial Sequence
  Template to Reverse primer
gggccatcaacggttt

SEQ ID NO: 20 - DNA, 17 bp, Artificial Sequence
  Template to Fluorescent labelled Probe
tttagcagcaccggtag

SEQ ID NO: 21 - DNA, 282 bp, Artificial Sequence
  Coliforms GAL coding lacZ gene sequences gi|126038352|gb|EF136884.1| Escherichia coli K-12 lac operon, complete sequence
cgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgt

SEQ ID NO: 22 - DNA, 24 bp, Artificial Sequence
  Template to Forward primer
atgaaagctggctacaggaaggcc

SEQ ID NO: 23 - DNA, 20 bp, Artificial Sequence
  Template to Reverse primer
ggaagatcaggatatgtggc

SEQ ID NO: 24 - DNA, 26 bp, Artificial Sequence
  Template to Fluorescent labelled Probe
cgtttgccgtctgaatttgacctgag

SEQ ID NO: 25 - DNA, 222 bp, Artificial Sequence
  Coliforms GUS coding uidA gene sequences gi|38201937|gb|AY447088.1| Escherichia coli beta-glucuronidase (uidA) gene, uidA-A42 allele, partial cds
aactgaactggcagactatcccgccgggcatggtaattaccgacgaaaacggcaagaaaaagcagtcttacttccatgatttctttaactacgccgggatacatcgcagcgtaatgctctacaccacgccgaacacctgggtggacgatatcaccgtggtgacgcatgttgcgcaagactgtaaccacgcgtctgttgactggcaggtggtggcaaatggtg

SEQ ID NO: 26 - DNA, 23 bp, Artificial Sequence
  Template to Forward primer
tggtaattaccgacgaaaacggc

SEQ ID NO: 27 - DNA, 21 bp, Artificial Sequence
  Template to Reverse primer
cgcaagactgtaaccacgcgt

SEQ ID NO: 28 - DNA, 26 bp, Artificial Sequence
  Template to Fluorescent labelled Probe
gtaatgctctacaccacgccgaacac

SEQ ID NO: 29 - DNA, 260 bp, Escherichia coli
gcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgggcgcaacccttatcctttgttgccagcggtccggccgggaactcaaaggagactgccagtgataaactggaggaaggtggggatgac

SEQ ID NO: 30 - DNA, 18 bp, Escherichia coli
gatgcaacgcgaagaacc

SEQ ID NO: 31 - DNA, 19 bp, Escherichia coli
gaactcaaaggagactgcc

SEQ ID NO: 32 - DNA, 20 bp, Escherichia coli
atgagaatgtgccttcggga

SEQ ID NO: 33 - DNA, 211 bp, Escherichia coli
ggttgcgaaggaatttaccttagacttctcgactgcaaagacgtatgtagattcgctgaatgtcattcgctctgcaataggtactccattacagactatttcatcaggaggtacgtctttactgatgattgatagtggcacaggggataatttgtttgcagttgatgtcagagggatagatccagaggaagggcggtttaataatctacgg

SEQ ID NO: 34 - DNA, 21 bp, Escherichia coli
gactgcaaagacgtatgtaga

SEQ ID NO: 35 - DNA, 21 bp, Escherichia coli
gttgatgtcagagggatagat

SEQ ID NO: 36 - DNA, 20 bp, Escherichia coli
caggaggtacgtctttactg

SEQ ID NO: 37 - DNA, 142 bp, Pseudomonas aeruginosa
cggcagcctgagcgacgaagccgctctgcgtgcgatcaccaccttctacttcgagtacgacagctccgacctgaagccggaagccatgcgcgctctggacgtacacgcgaaagacctgaaaggcagcggtcagcgcgtagtg

SEQ ID NO: 38 - DNA, 21 bp, Pseudomonas aeruginosa
tgcgatcaccaccttctactt

SEQ ID NO: 39 - DNA, 19 bp, Pseudomonas aeruginosa
tctggacgtacacgcgaaa

SEQ ID NO: 40 - DNA, 26 bp, Pseudomonas aeruginosa
agtacgacagctccgacctgaagccg

SEQ ID NO: 41 - DNA, 242 bp, Pseudomonas aeruginosa
acgccgcgcgcctgccgtcctggccgccggcgccgcaatacgagtatctggccgacagcatgtggccgcagttcgcctacgggcgcaacgcggtgtatcccgaaggccaccatggcaatgccgtgctctccaagcatccgatcctcgcccaccgcaacctcgacgtctcggtggcgggcaacgaggaacgcggcctgttgcatgcggtgatcgacatcggccgtccgctgcacgcggtctgc

SEQ ID NO: 42 - DNA, 18 bp, Pseudomonas aeruginosa
cgccgcaatacgagtatc

SEQ ID NO: 43 - DNA, 18 bp, Pseudomonas aeruginosa
ctgttgcatgcggtgatc

SEQ ID NO: 44 - DNA, 17 bp, Pseudomonas aeruginosa
ggcgggcaacgaggaac

SEQ ID NO: 45 - DNA, 193 bp, Enterococcus faecalis
cctaatacatgcaagtcgaacgcttctttcctcccgagtgcttgcactcaattggaaagaggagtggcggacgggtgagtaacacgtgggtaacctacccatcagagggggataacacttggaaacaggtgctaataccgcataacagtttatgccgcatggcataagagtgaaaggcgctttcgggtgtcgc

SEQ ID NO: 46 - DNA, 19 bp, Enterococcus faecalis
cgcttctttcctcccgagt

SEQ ID NO: 47 - DNA, 18 bp, Enterococcus faecalis
cagtttatgccgcatggc

SEQ ID NO: 48 - DNA, 25 bp, Enterococcus faecalis
caattggaaagaggagtggcggacg

SEQ ID NO: 49 - DNA, 337 bp, Enterococcus faecalis
cacaccaggtatgcctctatctgttgagttaaatgccgtgggtaatgtggttaaaattaatacaagtaaaaaagtacaattacctcatagtattccgatggaagtcgttgattttgatcttgaaaaagaattattcatcaagggctatgtcaatggaaacgaagaagaagaaaccgtttataaagttgaccatgatgcaacgattattgaaagtgatggaaccgaggtgcggattgcgccacttgacgttcaatttcaatcagcgaaattatcgcaacgcattttaacgaactttgcgggacccatgaataactttatcttagggtttattctgttt

SEQ ID NO: 50 - DNA, 20 bp, Enterococcus faecalis
aaatgccgtgggtaatgtgg

SEQ ID NO: 51 - DNA, 19 bp, Enterococcus faecalis
gaactttgcgggacccatg

SEQ ID NO: 52 - DNA, 21 bp, Enterococcus faecalis
gaaagtgatggaaccgaggtg

SEQ ID NO: 53 - DNA, 438 bp, Clostridium perfringens
cggggacccgcacaagtagcggagcatgtggtttaattcgaagcaacgcgaagaaccttacctacacttgacatcccttgcattactcttaatcgaggaaatccttcggggacaaggtgacaggtggtgcatggttgtcgtcagctcgtgtcgtgagatgttgggttaagtcccgcaacgagcgcaacccttgtcgttagttactaccattaagttgaggactctagcgagactgcctgggttaaccaggaggaaggtggggatgacgtcaaatcatcatgccccttatgtgtagggctacacacgtgctacaatggctggtacagagagatgcaataccgcgaggtggagccaaacttaaaaaccagtctcagttcggattgtaggctgaaactcgcctacatgaagctggagttactagtaatcgcgaatcagaat

SEQ ID NO: 54 - DNA, 20 bp, Clostridium perfringens
gcgaagaaccttacctacac

SEQ ID NO: 55 - DNA, 20 bp, Clostridium perfringens
gattgtaggctgaaactcgc

SEQ ID NO: 56 - DNA, 21 bp, Clostridium perfringens
aagttgaggactctagcgaga

SEQ ID NO: 57 - DNA, 533 bp, Clostridium perfringens
agatatagatactccatatcatcctgctaatgttactgccgttgatagcgcaggacatgttaagtttgagacttttgcagaggaaagaaaagaacagtataaaataaacacagcaggttgcaaaactaatgaggatttttatgctgatatcttaaaaaacaaagattttaatgcatggtcaaaagaatatgcaagaggttttgctaaaacaggaaaatcaatatactatagtcatgctagcatgagtcatagttgggatgattgggattatgcagcaaaggtaactttagctaactctcaaaaaggaacagcaggatatatttatagattcttacacgatgtatcagagggtaatgatccatcagttggaaagaatgtaaaagaactagtagcttacatatcaactagtggtgagaaagatgctggaacagatgactacatgtattttggaatcaaaacaaaggatggaaaaactcaagaatgggaaatggacaacccaggaaatgattttatgactggaagtaaagacactt

SEQ ID NO: 58 - DNA, 20 bp, Clostridium perfringens
tgttactgccgttgatagcg

SEQ ID NO: 59 - DNA, 20 bp, Clostridium perfringens
ggaaatggacaacccaggaa

SEQ ID NO: 60 - DNA, 21 bp, Clostridium perfringens
gtggtgagaaagatgctggaa

SEQ ID NO: 61 - DNA, 391 bp, Salmonella enterica
gaaactggcaggcttgagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtctacttggaggttgtgcccttgaggcgtggcttccggagctaacgcgttaagtagaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgcct

SEQ ID NO: 62 - DNA, 20 bp, Salmonella enterica
gggtagaattccaggtgtag

SEQ ID NO: 63 - DNA, 20 bp, Salmonella enterica
cttacctggtcttgacatcc

SEQ ID NO: 64 - DNA, 20 bp, Salmonella enterica
tgtctacttggaggttgtgc

SEQ ID NO: 65 - DNA, 144 bp, Salmonella enterica
agcgcgccggctttcaacgcctctaccgccgtttccacgctggaaaatgccgtcataatcaaaatcggaatggcgggattgagcgctttaatctccttcagcgtggcgataccgtccatctccgccatacgcacatcgcacagt

SEQ ID NO: 66 - DNA, 20 bp, Salmonella enterica
gtttccacgctggaaaatgc

SEQ ID NO: 67 - DNA, 19 bp, Salmonella enterica
cttcagcgtggcgataccg

SEQ ID NO: 68 - DNA, 22 bp, Salmonella enterica
tcggaatggcgggattgagcgc

SEQ ID NO: 69 - DNA, 330 bp, Staphylococcus aureus
cggtcttgctgtcacttatagatggatccgcgctgcattagctagttggtaaggtaacggcttaccaaggcaacgatgcatagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaatac

SEQ ID NO: 70 - DNA, 19 bp, Staphylococcus aureus
cgctgcattagctagttgg

SEQ ID NO: 71 - DNA, 20 bp, Staphylococcus aureus
cctaatcagaaagccacggc

SEQ ID NO: 72 - DNA, 24 bp, Staphylococcus aureus
cgtgagtgatgaaggtcttcggat

SEQ ID NO: 73 - DNA, 277 bp, Staphylococcus aureus
agcaaaacaaatgcatataacgtaacaacacatgcaaacggccaagtatcatacggagctcgcccgacatacaagaaaccaagcaaaacaaatgcatacaacgtaacaacacatgcaaatggtcaagtatcatatggcgctcgcccgacacaaaaaaagccaagcaaaacaaatgcatataacgtaacaacacatgcaaatggtcaagtatcatacggagctcgcccgacatacaagaagccaagcgaaacaaatgcatacaacgtaacaacacatg

SEQ ID NO: 74 - DNA, 20 bp, Staphylococcus aureus
catgcaaacggccaagtatc

SEQ ID NO: 75 - DNA, 20 bp, Staphylococcus aureus
gacatacaagaagccaagcg

SEQ ID NO: 76 - DNA, 21 bp, Staphylococcus aureus
cgcccgacatacaagaaacca

SEQ ID NO: 77 - DNA, 478 bp, Campylobacter jejuni
agcgtggggagcaaacaggattagataccctggtagtccacgccctaaacgatgtacactagttgttgggatgctagtcatctcagtaatgcagctaacgcattaagtgtaccgcctggggagtacggtcgcaagattaaaactcaaaggaatagacggggacccgcacaagcggtggagcatgtggtttaattcgaagatacgcgaagaaccttacctgggcttgatatcctaagaaccttatagagatatgagggtgctagcttgctagaacttagagacaggtgctgcacggctgtcgtcagctcgtgtcgtgagatgttgggttaagtcccgcaacgagcgcaacccacgtatttagttgctaacggttcggccgagcactctaaatagactgccttcgtaaggaggaggaaggtgtggacgacgtcaagtcatcatggcccttatgcccagggcgacacacgtgctacaatgg

SEQ ID NO: 78 - DNA, 19 bp, Campylobacter jejuni
ggtagtccacgccctaaac

SEQ ID NO: 79 - DNA, 20 bp, Campylobacter jejuni
gtcaagtcatcatggccctt

SEQ ID NO: 80 - DNA, 21 bp, Campylobacter jejuni
gaacttagagacaggtgctgc

SEQ ID NO: 81 - DNA, 274 bp, Campylobacter jejuni
ttttggtgtgattatgttggcaattcataataaagaacctaaaattttctctttgttggaacttttaaatcttttcttaactcatagaaaaacagttattattagaagaacgatttttgaacttcaaaaggcaagagcaagagctcatattttagaaggtcttaaaattgcacttgataatatagatgaagtgattgctttaattaaaaatagttctgataataataccgcaagagattctttagtagctaaatttggtcttagtgagcttcaa

SEQ ID NO: 82 - DNA, 29 bp, Campylobacter jejuni
taaagaacctaaaattttctctttgttgg

SEQ ID NO: 83 - DNA, 29 bp, Campylobacter jejuni
ctgataataataccgcaagagattcttta

SEQ ID NO: 84 - DNA, 29 bp, Campylobacter jejuni
ttcaaaaggcaagagcaagagctcatatt

SEQ ID NO: 85 - DNA, 672 bp, Listeria monocytogenes
gcgtagatatgtggaggaacaccagtggcgaaggcgactctctggtctgtaactgacgctgaggcgcgaaagcgtggggagcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctggagacagagctttcccttcggggacaaagtgacaggtggtgcatggttgtcgtcagctcgtgtcgtgagatgttgggttaagtcccgcaacgagcgcaacccttgattttagttgccagcatttagttgggcactctaaagtgactgccggtgcaagccggaggaaggtggggatgacgtcaaatcatcatgccccttatgacctgggctacacacgtgctacaatggatgattttagttgccagcatttagttgggcactctaaagtgactgccggtgcaagccggaggaaggtggggatgacgtcaaatcatcatgccccttatgacctgggctacacacgtgctacaatggat

SEQ ID NO: 86 - DNA, 20 bp, Listeria monocytogenes
aaggcgactctctggtctgt

SEQ ID NO: 87 - DNA, 22 bp, Listeria monocytogenes
gtcaaatcatcatgccccttat

SEQ ID NO: 88 - DNA, 21 bp, Listeria monocytogenes
ccgcaaggttgaaactcaaag

SEQ ID NO: 89 - DNA, 350 bp, Listeria monocytogenes
taaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggt

SEQ ID NO: 90 - DNA, 19 bp, Listeria monocytogenes
gcgcttggagtgaatgcag

SEQ ID NO: 91 - DNA, 19 bp, Listeria monocytogenes
gatgctgccgtaagcggaa

SEQ ID NO: 92 - DNA, 20 bp, Listeria monocytogenes
ggcgtatggccgtcaagttt

SEQ ID NO: 93 - DNA, 559 bp, Shigella flexneri
tgtctcatccagcacgtccgggtaaggcaggaaggcgttcgctacaattcgcagctcgccgccgctattaagatgacgcaccgcgccacgaatcagcgtttgcgccgcatccaggctggtttgcatcccatcgtggaacggcgggttggagatgatcatatcaaaacaacctttcacctcggaaaagacgttgctggcaaagacttcaccttcaacaccgttggctgcaagtgttgcgcggctggcttctaccgccggggcagagacatcgcacaaggtgagacgaattttcggcgaatggcgcgcaaaggcaactgaaagtacacccgcgccacagccgacatccagcactttacctttcgtgtgcggagttaacgtcgagagcagcaactggctaccgacatccagaccgtcgcggctaaacacgccaggcagcgttttgaccgtcaggccatcgacgctgtattcgccccagaatttatccgcatcaaatactggctgtttttccagacgaccaaaatagaggccacagcgacgagcgctgtcgactttattcaac

SEQ ID NO: 94 - DNA, 20 bp, Shigella flexneri
gaaggcgttcgctacaattc

SEQ ID NO: 95 - DNA, 20 bp, Shigella flexneri
gacgaccaaaatagaggcca

SEQ ID NO: 96 - DNA, 22 bp, Shigella flexneri
catatcaaaacaacctttcacc

SEQ ID NO: 97 - DNA, 266 bp, Shigella flexneri
atatctcaggggaccacatcggtgtctgttattaaccacaccccaccgggcagttattttgctgtggatatacgagggcttgatgtctatcaggcgcgttttgaccatcttcgtctgattattgagcaaaataatttatatgtggccgggttcgttaatacggcaacaaatactttctacagattttcagattttgcacatatatcagtgcccggtgtgacaacggtttccatgacaacggacagcagttataccactctgcaacg

SEQ ID NO: 98 - DNA, 18 bp, Shigella flexneri
attaaccacaccccaccg

SEQ ID NO: 99 - DNA, 20 bp, Shigella flexneri
gtgacaacggtttccatgac

SEQ ID NO: 100 - DNA, 19 bp, Shigella flexneri
gctgtggatatacgagggc

SEQ ID NO: 101 - DNA, 330 bp, Artificial Sequence; identical to SEQ ID NO: 69
  MRSA methicillin resistant Staphylococcus Aureus SA16S RNA coding sa16s-rna gene sequences NCBI Reference Sequence NC_009632.1 gi|150392480541500-543044 Staphylococcus aureus subsp. aureus JH1, complete genome

SEQ ID NO: 102 - DNA, 19 bp, Artificial Sequence
  Template to Forward primer
cgctgcattagctagttgg

SEQ ID NO: 103 - DNA, 19 bp, Artificial Sequence
  Template to Reverse primer
cctaatcagaaagccacgg

SEQ ID NO: 104 - DNA, 24 bp, Artificial Sequence
  Template to Fluorescent labelled Probe
cgtgagtgatgaaggtcttcggat

SEQ ID NO: 105 - DNA, 435 bp, Artificial Sequence
  MRSA methicillin resistant Staphylococcus Aureus PBP2a coding mecA gene sequences gi|186966530|gb|EU437549.1| Staphylococcus aureus strain cm11 SCCmec type IVA element, complete sequence; and hypothetical
acaacatgaaaaatgattatggctcaggtactgctatccaccctcaaacaggtgaattattagcacttgtaagcacaccttcatatgacgtctatccatttatgtatggcatgagtaacgaagaatataataaattaaccgaagataaaaaagaacctctgctcaacaagttccagattacaacttcaccaggttcaactcaaaaaatattaacagcaatgattgggttaaataacaaaacattagacgataaaacaagttataaaatcgatggtaaaggttggcaaaaagataaatcttggggtggttacaacgttacaagatatgaagtggtaaatggtaatatcgacttaaaacaagcaatagaatcatcagataacattttctttgctagagtagcactcgaattaggcagtaagaaatttgaaaaaggca

SEQ ID NO: 106 - DNA, 19 bp, Artificial Sequence
  Template to Forward primer
ctgctatccaccctcaaac

SEQ ID NO: 107 - DNA, 20 bp, Artificial Sequence
  Template to Reverse primer
ctttgctagagtagcactcg

SEQ ID NO: 108 - DNA, 24 bp, Artificial Sequence
  Template to Fluorescent labelled Probe
gaacctctgctcaacaagttccag

SEQ ID NO: 109 - DNA, 391 bp, Legionella pneumophila
cggtaatacggagggtgcaagcgttaatcggaattactgggcgtaaagggtgcgtaggtggttgattaagttatctgtgaaattcctgggcttaacctgggacggtcagataatactggttgactcgagtatgggagagggtagtggaatttccggtgtagcggtgaaatgcgtagagatcggaaggaacaccagtggcgaaggcggctacctggcctaatactgacactgaggcacgaaagcgtggggagcaaacaggattagataccctggtagtccacgctgtaaacgatgtcaactagctgttggttatatgaaaataattagtggcgcagcaaacgcgataagttgaccgcctggggagtacggtcgcaagattaaaactcaaagg

SEQ ID NO: 110 - DNA, 20 bp, Legionella pneumophila
gaattactgggcgtaaaggg

SEQ ID NO: 111 - DNA, 18 bp, Legionella pneumophila
gataagttgaccgcctgg

SEQ ID NO: 112 - DNA, 25 bp, Legionella pneumophila
gggacggtcagataatactggttga

SEQ ID NO: 113 - DNA, 304 bp, Legionella pneumophila
tagatgttaatccggaagcaatggctaaaggcatgcaagacgctatgagtggcgctcaattggctttaaccgaacagcaaatgaaagacgttcttaacaagtttcagaaagatttgatggcaaagcgtactgctgaattcaataagaaagcggatgaaaataaagtaaaaggggaagcctttttaactgaaaacaaaaacaagccaggcgttgttgtattgccaagtggtttgcaatacaaagtaatcaattctggaaatggtgttaaacccggaaaatcggatacagtcactgtcgaatacac

SEQ ID NO: 114 - DNA, 19 bp, Legionella pneumophila
gcatgcaagacgctatgag

SEQ ID NO: 115 - DNA, 20 bp, Legionella pneumophila
ggaaatggtgttaaacccgg

SEQ ID NO: 116 - DNA, 20 bp, Legionella pneumophila
acaagccaggcgttgttgta

SEQ ID NO: 117 - DNA, 228 bp, Artificial Sequence
  Mycobacterium tuberculosis complex MTB16S RNA coding mtb16s-rna gene sequences gi|44689|emb|X52917.1| Mycobacterium tuberculosis 16S rRNA gene
gaattactgggcgtaaagagctcgtaggtggtttgtcgcgttgttcgtgaaatctcacggcttaactgtgagcgtgcgggcgatacgggcagactagagtactgcaggggagactggaattcctggtgtagcggtggaatgcgcagatatcaggaggaacaccggtggcgaaggcgggtctctgggcagtaactgacgctgaggagcgaaagcgtggggagcgaacag

SEQ ID NO: 118 - DNA, 19 bp, Artificial Sequence
  Template to Forward primer
gtttgtcgcgttgttcgtg

SEQ ID NO: 119 - DNA, 19 bp, Artificial Sequence
  Template to Reverse primer
ctctgggcagtaactgacg

SEQ ID NO: 120 - DNA, 20 bp, Artificial Sequence
  Template to Fluorescent labelled Probe
aattcctggtgtagcggtgg

SEQ ID NO: 121 - DNA, 310 bp, Artificial Sequence
  Mycobacterium tuberculosis complex MTC IS6110 coding is6110 gene sequences gi|2342529|emb|Y14613.1| Mycobacterium tuberculosis ipl7::IS6110 IS-like element
gagctgggtgtgccgatcgccccatcgacctactacgaccacatcaaccgggagcccagccgccgcgagctgcgcgatggcgaactcaaggagcacatcagccgcgtccacgccgccaactacggtgtttacggtgcccgcaaagtgtggctaaccctgaaccgtgagggcatcgaggtggccagatgcaccgtcgaacggctgatgaccaaactcggcctgtccgggaccacccgcggcaaagcccgcaggaccacgatcgctgatccggccacagcccgtcccgccgatctcgtccagcgccgcttcg

SEQ ID NO: 122 - DNA, 20 bp, Artificial Sequence
  Template to Forward primer
tactacgaccacatcaaccg

SEQ ID NO: 123 - DNA, 20 bp, Artificial Sequence
  Template to Reverse primer
cgctgatccggccacagccc

SEQ ID NO: 124 - DNA, 24 bp, Artificial Sequence
  Template to Fluorescent labelled Probe
caaagtgtggctaaccctgaaccg

(19) United States
(12) Patent Application Publication
(10) Pub. No.: US 2013/0324436 A1
(43) Pub. Date: Dec. 5, 2013

(54) PROCEDURE FOR NUCLEIC ACID-BASED DIAGNOSTIC DETERMINATION OF BACTERIAL GERM COUNTS AND KIT FOR THIS PURPOSE

(71) Applicants: Gabor Kiss, Szamosszeg (HU); Janos Kiss, Szamosszeg (HU); Timea Kiss, Szamosszeg (HU); Ambrusne Sztancsik Katalin Kovacs, Budapest (HU); Georgina Bernath, Budapest (HU)

(72) Inventors: Gabor Kiss, Szamosszeg (HU); Janos Kiss, Szamosszeg (HU); Timea Kiss, Szamosszeg (HU); Ambrusne Sztancsik Katalin Kovacs, Budapest (HU); Georgina Bernath, Budapest (HU)

(73) Assignee: DIAGON KFT, Budapest (HU)

(21) Appl. No.: 13/990,242

(22) PCT Filed: Nov. 30, 2010
(86) PCT No.: PCT/HU10/00132

§ 371 Date: May 29, 2013

(51) Int. Cl. C12Q 1/68

(52) U.S. Cl. CPC C12Q 1/689
USPC 506/9; 435/6.11; 506/16

(57) ABSTRACT

Disclosed are procedures and kits for nucleic acid-based molecular diagnostic determination of bacterial germ counts during which procedure evolutionarily conserved genes and genes coding for characteristic pathogenicity markers, favourably microbial enzyme, toxin, special resistance, are detected using real-time PCR amplification method with the application of fluorescent hydrolysis probes. The multiplication of nucleotide chains takes place with oligonucleotides annealing to the structural gene 5′ end region and to the adjacent upstream regulatory promoter-operator region so that the presence of the structural gene is shown along with the adjacent upstream regulatory promoter-operator sequences; the functional nature of the structural gene is simultaneously checked. The result is measured with a genome unit equivalent DNA amount calibrated to the germ number of sample units equivalent to standard procedures. The calibrated determination of bacterial germ counts is favourably based on single copy gene sequences in the genome, like those coding for characteristic pathogenicity markers.

The subject of the invention relates to a procedure for nucleic acid-based molecular diagnostic determination of bacterial germ counts using real-time PCR amplification method, with the help of fluorescent hydrolysis probes. The invention also relates to KITs serving for the practical implementation of the procedure.

In the nucleic acid-based molecular diagnostic determination of bacterial germ counts we detect evolutionarily conserved genes and genes coding for characteristic pathogenicity markers, favourably microbial enzyme, toxin, special resistance in such a way that DNA chains amplified contain the structural genes along with the adjacent upstream regulatory promoter-operator sequences as a result of priming oligonucleotides annealing to the structural gene 5′ end region and to the adjacent upstream regulatory promoter-operator sequences. The PCR amplification result according to our method is measured by GU genome unit equivalent to the amount of DNA calibrated to the CFU germ count of the sample unit defined in standard procedures. The calibrated determination according to our procedure of bacterial germ counts is favourably based on single copy gene sequences in the genome, like those coding for characteristic pathogenicity markers. With our quantifiable simple procedure that can be measured by instrument we make it possible to quickly determine the bacterial germ count from drinking water, food products and from further hygiene and clinical samples.

During the public hygienic monitoring of drinking waters, environmental waters, food products, healthcare working areas, communal and environmental surfaces an essential parameter of microbial tests is the qualitative and quantitative determination of the colony-forming units of indicator bacterial germs. To make these determinations in microbial practices standardised, time-consuming culturing procedures are used at present. The alternative, nucleic acid-based, biochemically specific molecular diagnostic procedure described in our invention serves to supplement and/or replace the aforementioned standard technique by giving a quicker result as compared to standard methods and by having sufficiently reliable, well defined technical-measurement characteristics. Furthermore, the subject of the invention also relates to the generalisation of the procedures serving to identify bacteria detailed in the specification into a nucleic acid based molecular diagnostic procedure through which—with favourable conditions similar to the detailed procedures—the presence of the searched-for structural genes in biological or other samples may be determined in a quantitative way much faster than the known solutions. The KITs planned for the application serve for the practical realisation of all this.

The term PCR (polymerase chain reaction) indicates the in vitro multiplication, enzymatic amplification of the nucleic acids carrying the genetic material [for details of PCR method see U.S. Pat. Nos. 4,889,818, 4,683,195, 4,683,202, 4,965,188]. In practice the traditional PCR thermocycling reaction is the in vitro cyclic repetition of nucleic acid replication, as a result of which the examined double stranded DNA doubles every cycle. The start of the individual cycles is heat denaturation Tm melting temperature to separate the antiparallel orientation nucleotide strands of double stranded DNA into single DNA strands. Then, at different temperature, on these antiparallel, separated single DNA strands i.e. DNA templates specific, short, complementary PCR probe elements, oligonucleotide primers hybridize in the upstream and downstream positions delimiting the region to be identified. These oligonucleotide primers, i.e. primer pairs of forward and reverse primers hybridizing in the delimiting positions as above initiate and delimit the in vitro enzymatic amplification, the multiplication of the DNA template region to be identified. The aforementioned in vitro enzymatic amplification, multiplication of the DNA template region to be identified takes place in the presence of heatstable DNA polymerase, nucleotides and buffer components (e.g. ions, organic bases). This thermocycling reaction takes place in cycles repeated several times and has a time-temperature profile characteristic for the nucleotide sequence of the template. In the DNA template region to be identified the nucleotide sequence of the template double in every cycle according to the 2ⁿ algorithm. After 25-35 cycles the multiplied nucleotide chains may be separated according to size with traditional separation technology, horizontal gel electrophoresis and may be made visible by densitometry or staining, and, furthermore, may be determined by sequencing. When showing the presence of RNA sequences, prior to the PCR process a reverse transcription takes place, in which a cDNA complementary DNA chain is produced from the RNA template, and this cDNA complementary DNA enters the cyclic process of enzymatic amplification according to the above.

Real-time PCR methods appeared to shorten duration time of tests comprising the traditional thermocycling reaction+separation techniques and are gaining more and more ground in the new millennium. In real-time PCR methods the enzymatic amplification of the DNA template region delimited by forward and reverse primers is detected with the help of fluorescent labelled oligonucleotides, oligo probes. The so-called internal probes are fluorescent labelled oligo probes that hybridize to the complementary sequences of the DNA template region delimited for enzymatic amplification. In the case of so-called hydrolysis probes the fluorescent labelled oligo probes hybridized to the complementary sequences of the DNA template region delimited for enzymatic amplification are cleaved from the template by hydrolysis due to heatstable DNA polymerase exonuclease activity during chain elongation. Hereby, the fluorescent signal released every amplification cycle is to be registered in real-time measurement [see U.S. Pat. Nos. 5,210,015, 5,487,972, 5,804,375, 6,214,979]. The progress of the reaction is to be monitored by measuring the intensity of the fluorescence, then at the end of the reaction the kinetics may also be demonstrated. By monitoring reaction kinetics we may obtain precise mathematic information on the global kinetic parameters of the reaction. The advantages of real-time PCR is that in quantitative determinations it provides data approaching initial DNA template concentration the best with its Cp crossing point (fluorescence intensity that exceeding the background value shows the presence of the searched-for template) and its Ct cycle threshold (cycle number at which the intensity of fluorescence exceeding the background value shows the presence of the searched-for template) values. The Cp crossing point and Ct cycle threshold values represent the kinetic state when the amplification reaction enters the exponential phase, when the fluorescence intensity measured is the most in proportion with the initial amount of template DNA. Contrary to all this, the traditional thermocycling+separation technique detailed earlier only makes it possible to measure the endpoint of the amplification reaction.

It is characteristic of the various PCR methods that the way of detecting nucleic acids targeted may be simplex or multiplex. The simplex way serves for the detection of a target sequence of the template region delimited for enzymatic amplification [for example invention U.S. Pat. No. 5,795,717]. The multiplex way makes it possible to detect target sequences (multitargeted testing) of several template regions delimited for enzymatic amplification in a single reaction space and at the same time [for example, the multiplex PCR amplification detecting of drinking water E. coli and Clostridium perfringens with the help of lacZ-uidA and p/c gene sequences in Tantawiwat S. et al. (2005): Southeast Asian J. Trop. Med. Public Health 36: 162-169, or the detection of Enterobacteriaceae, E. coli virulence factors in specification no. WO 03052143, furthermore multitargeted testing in patent specifications nos. WO 0146477 and WO 2008074023, as well as multitargeted assay statistical reliability in patent specification no. WO 2005103284].

The advantage of real-time PCR devices is that the multiplex way results received by oligo probes labelled by various techniques may be detected in a single reaction space with fluorescent channels operating at various wavelengths at the same time. These are the so-called multiplex-multicolour techniques. In the procedure according to the present specification we used duplex amplification conforming to template regions of two target genes and a dual colour fluorescent signaling system.

The fluorophores used in real-time PCR methods may be in covalent bond with various combinations of oligonucleotide primers, probes [for example, self-quenching fluorescence probe in U.S. Pat. No. 5,538,848, furthermore, fluorescent labelled oligos in U.S. Pat. Nos. 5,723,591 and 5,876,930, or different from these double stranded DNA binding fluorescent dye in the amplification mixture in U.S. Pat. No. 6,171,785]. In our procedure we used hydrolysis oligonucleotide probes (see earlier) without restricting the scope of protection to only this type of oligonucleotide probe.

We present our nucleic acid based molecular diagnostic procedure elaborated for the determination of bacterial germ counts in hygiene samples of drinking waters, environmental waters, food products, healthcare working areas, communal and environmental surfaces with the following indicator organisms: total heterotrophic plate count, Coliforms, E. coli, Pseudomonas aeruginosa, Enterococcus faecalis, Clostridium perfringens, Salmonella enterica, Staphylococcus aureus, Campylobacter jejuni and coli, Listeria monocytogenes, Shigella flexneri, Methicillin resistant Staphylococcus aureus, Legionella pneumophila, Mycobacterium tuberculosis.

The present standard methods (EN-ISO 6222, EN-ISO 7899-2, EN-ISO 9308-1, EN-ISO 9308-2, EN-ISO 12780, EN-ISO 16140, EN-ISO 17994, EN-ISO 26461-2) used for the distinctive detection of indicator bacteria listed are the traditional culturing methods performed with the help of membrane filtration or MPN (Most Probable Number—with dilution series) techniques, which are performed in nonselective and selective culturing media, and the result is evaluated on the 3^{rd}-10^{th} day following inoculation, in other words the number of bacterial colonies that have grown in the medium is determined. The advantage of these methods is that they are standard applications that are accepted all over the world, and with respect to the demand for chemicals, equipment and other infrastructure they are cheap solutions. The disadvantage of these methods is that they are very time-consuming, and characteristically of all culturing techniques, environmental effects have a significant influence on the process. A further disadvantage they have is that in the genus-species level detection of a given bacterium the efficiency of these culturing methods is questionable. Therefore the optimization of the procedure is difficult, and as instrumental evaluation is not involved, the human factors have a significant impact on the result.

A further possibility for the distinctive detection of indicator bacteria is the immunological detection of characteristic pathogenicity markers, like, for example, toxin production, or cell surface determinants. For example, patent specification no. WO 9628731 proves the presence of the E. coli EHEC strain with an antibody specific for Shiga-like toxin, or, for example, patent specification no. WO 2003106697 detects Pseudomonas aeruginosa contamination by the immune-agglutination of cell surface lipoprotein determinants. With these immunological techniques the detection is undoubtedly specific, but they do not encompass the cell populations expressing the examined markers-determinants partially or even modified.

From that stated till now it may be seen that the critical time factor (for example, culturing requiring several days) and the critical expression changes (for example, the immunological detectability of characteristic determinants) in the detection and quantitation of indicator bacteria, may be both more favourably handled with nucleic acid based solutions. One of the basic pillars of these latter solutions is the specific PCR amplification of evolutionarily conserved DNA sequences. Patent specification no. WO 0017381, for example, builds the detection of Bartonella and patent specification no. WO 9015157 builds the detection of Gram positive and Gram negative indicator bacteria on primer sequences specific for the regions coding 16S and 23S rRNA ribosomal ribonucleic acid. Patent specification no. WO 0059918 bases the testing of drug effect spectrum on the detection of Eubacteria tmDNA i.e. transfer-messenger DNA regions, similarly to patent specification no. WO 2006119466, which with the application of cDNA chip detecting evolutionarily conserved, taxonomically conserved rRNA gene regions accumulating local mutations, proves the presence of pathogenic Eubacteria (e.g. Enterobacteriaceae) in clinical samples.

For the specific quantitative determination of evolutionarily closely related species the detection of the aforementioned conserved DNA sequences is a necessary, but not a sufficient condition. In the invention recognition no. WO 0077242 the specific nutrient substrate composition of the in vitro culturing medium makes possible the selective separation of Salmonella, Shigella, E. coli 0157. In specification no. WO 03035889 the nucleic acid based detection of living bacterial cell population takes place with the help of selective typing bacteriophage. Generalising the above it may be determined that besides the evolutionarily conserved DNA sequences, taxonomical markers of evolutionarily closely related organisms the detection of sequence versions coding the distinctive markers characteristic of the species gives a much greater precision in the detection and quantitation of indicator bacteria. An example of this is patent specification no. WO 2007114509, which reports on oligo probes specific to Clostridium perfringens sequences in an immobilised DNA chip detection system. Another example is patent specification no. WO 2007076143, which presents primer pairs suitable for the detection of variable target sequences using a genome fragment enrichment (GFE) hybridization method, as well as specification no. WO 0112853, which carries out the detection of indicator bacteria with primers specific to the E. coli LamB and Enterococcus faecalis transposase Tn1546 gene sequences.

Summarising the aforementioned detection methods it may be determined that in the public health—hygiene determination of bacterial germ counts of various samples there is lacking a comprehensive instrumental analytical system that may be easily repeated, that supplements the present microbiological standard, that has the same value of this as regards its technical data, specificity and result and that exceeds its speed, and which involves the conditions described above.

When detecting any pathogenic bacterium which detection contains the above conditions, a further key issue of the reliable nucleic acid based quantitation of germ count is the choice of the characteristic template sequence.

The PCR study carried out by Franklin M. A. et al. [J. Vet. Diagn. Invest. (1996), 8: 460-463.] analyses the pathogenic adhesion-colonisation structural component of E. coli strains, the three expression variants of the gene coding the K88 fimbrial adhesin large subunit (K88ab, K88ac, K88ad strains) with the help of primers constructed for common and individual target sequences. The PCR products produced with 21 bp primer pairs are homologous with the common 764 bp internal operon region in all three antigen variants. The 24 bp primers specific for the expression variants hybridize upstream as compared to the 21 bp primers and as a result of the PCR reactions performed with the 21 bp-24 bp primer groups they detected nucleic acid sequences characteristic of the K₈₈ operon expression variants. In patent specifications no. WO 03000935 and no. US20060240442 they solved the detection of the E. coli O157:H7 variant in a food product sample after extracting the microbe DNA, with real-time PCR amplification of the region between the 1179-1539 nucleotides of the aea (attaching-effacing) gene that codes the cell surface pathogenic intimin protein. The detection was accomplished with 3′ fluorescein marked and 5′LCRed640-marked (FRET) internal hybridizing probes attached within a distance of six nucleotides. This train of thought was followed in the specific detection of Listeria monocytogenes (genome region between nucleotide bases 2987-3203) and Salmonella species (sipB-sipC region between nucleotide bases 2305-2555) as well.

In patent procedure no. WO 2007115590 they show Bacteroidetes infection originating from human or ruminant sources of environmental samples (e.g. water, faeces) with primers constructed for the species-specific sequence segments of the microbial 16S rRNA gene and, among others, with the help of fluorescent probes. The detection, i.e. the proving of infection originating from human or ruminant sources, did not take place in one, but with quantitative analysis evaluating two real-time PCR reactions. Differing from the above patent procedure no. EP 1,895,014 describes the multiplex PCR detection of Salmonella enterica Group I. serovariants in a one-tube solution. The system built on probes complying on European standards, operating according to various techniques (Taqman hydrolysis probe, Molecular beacon probe, Scorpion probe) is sensitive and specific for gene sequences that are taxonomically conserved and that code unique pathogenicity markers of the pathogenic bacteria. Patent procedure no. WO2004092406 proves Listeria monocytogenes content of samples with the application of various internal probes (see earlier description) detecting the presence of hlyA gene sequences that code endotoxin.

In the HACCP microbial hygienic monitoring of food products, primarily in samples of meats and dairy products monoplex and multiplex reactions [Maurer J. (ed): PCR Methods in Foods, pp. 62-64, 69-72, 77-78, 82-85. Springer-Birkhäuser, 2006] based on automated, tablet reagent systems proved the presence of Salmonella enteritidis, Listeria monocytogenes, E. coli O157:H7, Clostridium perfringens, etc. with, among others, CYBRGreen DNA intercalating fluorescent dye, or with a fluorescent TaqMan hydrolysis probe. The microbial target sequences of the detection include regions that code the pathogenicity markers (e.g. E. coli-eae gene, Listeria-hly gene), regulate transcription and code 16S rRNA. Syed Riyaz-Ul-Hassan et al. detect the E. coli STEC strain (shigatoxin coding stx gene and glucuronidase coding uidA gene) and the genetically similar Shigella (invasion plasmid antigen H coding ipaH gene) with triplex detecting, primer pairs producing PCR products of different length but with the same Tm (see earlier DNS heat denaturation at melting temperature) character [Syed Riyaz-Ul-Hassan et al.: J. Dairy Res. (2009), 76: 188-194.]. In this diagnostic solution the target is the structural gene coding the pathogenicity marker microbial toxin.

The taxonomic marker evolutionarily conserved genes and the genes coding the pathogenicity markers are detected separately in each case of the studies listed above, or the PCR reactions serving joint (e.g. single tube, single space) detection are specific for the so-called internal coding regions of the coding genetic elements (ORF—open reading frame, structural gene). The results of reactions of this type, like, for example, the fluorescent signal indicating the amplified nucleic acid region are not certain to show the functional presence of the gene coding the examined character.

Data originating from numerous attested bioinformation, international genetic databases (www.ncbi.nlm.nih.gov, www.embl.org, www.jdb.com) prove that the mere presence of a structural gene in tested genome does not necessarily mean that it is functional. This functionality depends on numerous factors. For example, on the basis of information gained from the genetic databases mentioned it can be observed that the presence of the structural gene in several cases does not mean the presence of the adjacent upstream regulatory elements that promote-operate the gene, in many cases the detectable enzyme activity is missing. During our experiments with several comparative measurements of the PCR and the traditional methods and with the help of bioinformation models we were successful in showing that several strains not exhibiting enzyme activity (e.g. Coliforms GAL/GUS galactosidase/glucuronidase) carry the structural genes that code the enzymes but they do not carry the sequences that regulate them.

On considering our above experiences in our own solution we started from the fact that in the case of the presence of the structural gene the biosynthesis of the pathogenicity marker coded by it may be questionable, for example, the biosynthesis of the microbial enzyme, toxin, resistance factor, in other words the presence of the adjacent upstream regulatory sequences promoting-operating the structural genes may be questionable. Because of this, in our amplification method we detect the examined structural genes along with the adjacent upstream regulatory promoter-operator sequences. As one of the elements of the PCR probe targeted at the joint detection we use the sequences of the structural gene 5′ end region and the sequences of the adjacent upstream regulatory promoter-operator as the other element. In our procedure when detecting target gene sequences coding the taxonomic marker and target gene sequences coding the pathogenicity markers favourably microbial enzyme, toxin, special resistance, the structural genes amplified with the sequences of the adjacent upstream regulatory promoter-operator on the basis of preliminary bioinformation analysis has greater reliability of avoiding false positive results caused by bacteria carrying non-functioning genes (deficient, deleted, or without regulatory region).

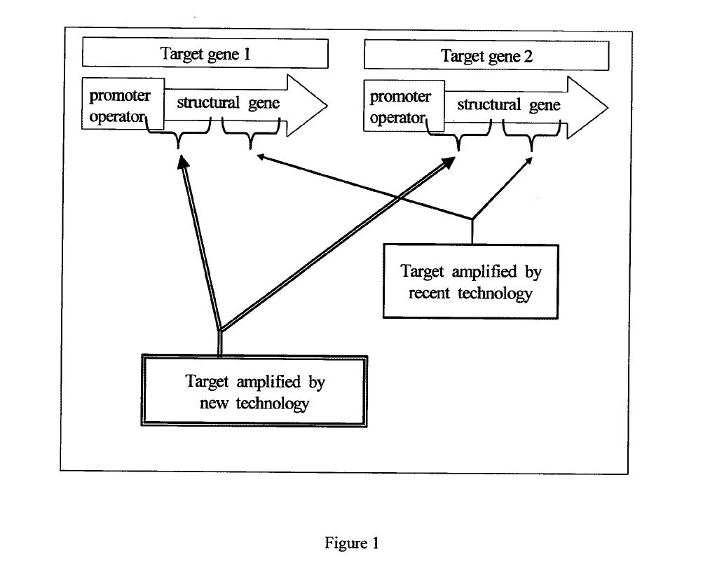

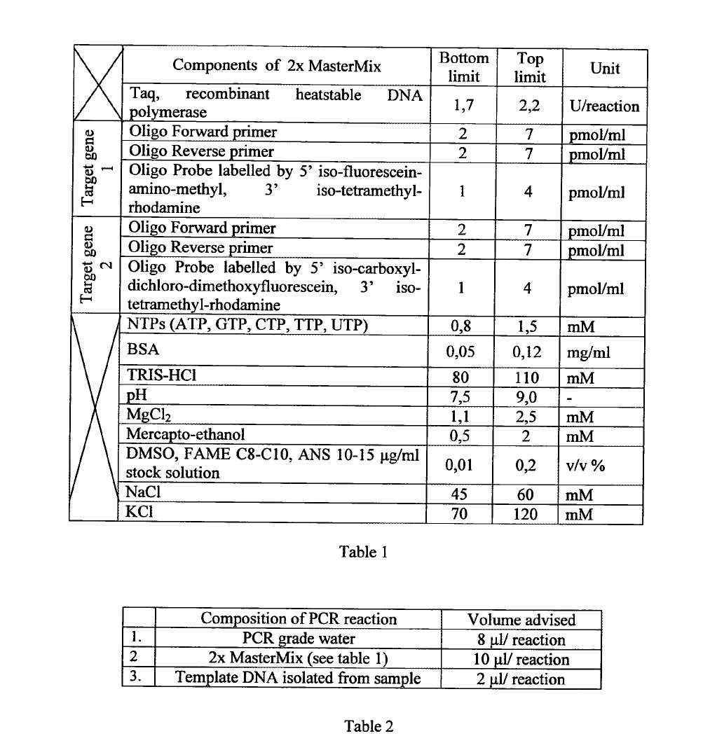

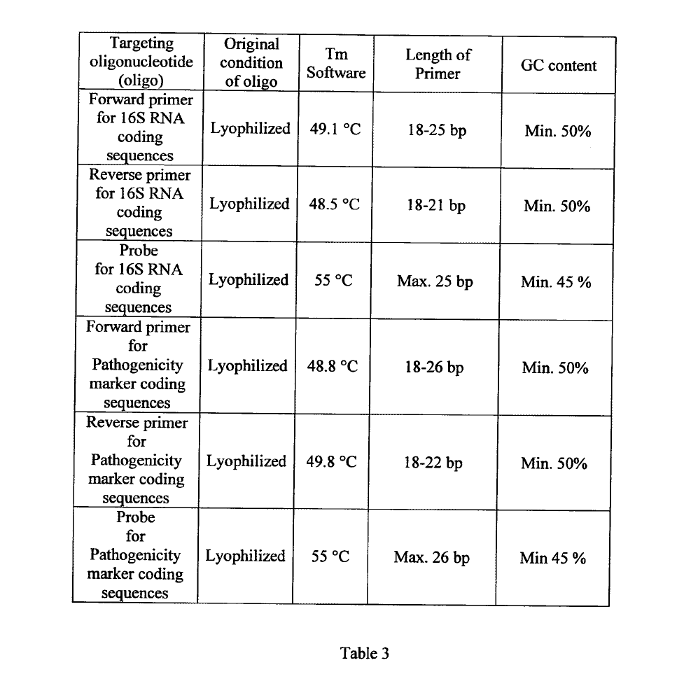

Summarising the above, for the detection of target gene sequences of indicator bacteria we have elaborated a duplex, dual colour technique that, taking the considerations described earlier, makes possible the determination of bacterial germ counts of public health, clinical and other hygiene samples with great reliability. A schematic drawing of a typical solution of our procedure may be seen on FIG. 1 (details at the description of the figure).

With our duplex, dual colour technique according to the invention procedure we detect the evolutionarily conserved 16S RNA coding sequences of certain indicator bacteria and the sequences coding their characteristic pathogenicity markers favourably enzyme, toxin, resistance, i.e. we detect sequences coding taxonomic and pathogenicity markers favourably with the sequences of the adjacent upstream regulatory promoter-operator region, in a PCR reaction, with the help of fluorescent hydrolysis probes.

We realised that due to the additives optimised by us (DMSO dimethyl sulfoxide, FAME fatty acid methyl ester C8-C10 fraction, ANS amino-naphtalenyl-sulfonic acid) the fluorescence spectrum of the fluorescent dyes of our dual colour technique may be effectively widened. With this Fluorescence Shift widening towards the red part of the spectrum our solution is much more reliable from the point of view of quantitative assessment, because instead of the emission spectrum given by certain real-time PCR dyes of 10-15 nm they give an emission maximum in a wavelength range 20-35 nm wider. Due to this the system may be used on most of the PCR platforms available on the market (Roche, ABI, BioRad, Corbett, Stratagene, etc.) with the desired precision, providing a reaction with excellent fluorescence characteristics, i.e. the specific fluorescence intensity (Δψ/Δt) is high during the measurement period.

The fluorescence labelling system of our duplex, dual colour technique is favourably iso-fluorescein-amino-methyl+iso-tetramethyl-rhodamine, and iso-carboxyl-dichloro-dimethoxyfluorescein+iso-tetramethyl-rhodamine, which are provided in the 2× concentrated MasterMix reaction mixture as the medium of our PCR reaction (for composition see table 1).

We have also observed that among the methods used in practice or disclosed to date there are none that use authentic, validated DNA standards equivalent to the traditional standard (EN-ISO) procedures for the calibration of the measurement. This is lacking from the range on offer by the PCR reagent developers and manufacturers, and in this way the result obtained by quantitative real-time PCR measurements is difficult to make compatible with the measurement range of culturing procedures of traditional public health, clinical practices. Therefore, in our invention procedure we have carried out the calibration of the measurements and drawn up the standards in such a way that in the case of a natural sample following 16 h selective enrichment culturing in the standard culture medium the GU genome unit equivalent of isolated bacterial DNA per standard sample unit is the same as the result received with the traditional membrane filtration colony counting carried out on the same sample, as the number per sample unit of the CFUs, the germ count. In other words, in the 3 calibration points required for the performance of authority measurements the GU genome unit equivalent DNA amount is equivalent to the 1000 CFU or 100 CFU or 10 CFU identical bacteria found in 1 ml or 1 g of standard sample unit, furthermore, to the 100 CFU or 10 CFU or 1 CFU identical bacteria found in 100 ml or 100 g of standard sample unit.

With the duplex, dual colour technique according to our procedure the detection of bacterial germs takes place with the joint detection of the two gene regions (see FIG. 1) detailed below of the indicator organisms listed previously.
In our invention procedure the HPC total Heterotrophic Plate Count designation is a collective name for Gram negative and Gram positive bacterial and also fungal microorganisms, which may be cultured in BHI Brain Heart Infusion general culture medium, under aerobic conditions at temperatures of 37° C. (HPC37), 30° C. (HPC30), and 22° C. (HPC22). Characteristically these most frequently include species of bacteria belonging to the genus Escherichia, Bacillus, Cytrobacter, Enterococcus, Enterobacter, Micrococcus, Lactobacillus, Salmonella, Staphylococcus etc., and numerous fungi (Aspergillus, Candida, Cladosporium, Fusarium, Saccharomyces). The two gene regions containing the target sequences of our HPC bacterial detection are the general microbial 16S RNA core coding core16s-rna and the GAPDH glyceraldehyde phosphate dehydrogenase enzyme coding gapdh.

In our invention procedure the CF Coliforms bacterial germs designation includes Gram negative microorganisms with GAL+/GUS+ galactosidase and glucuronidase enzyme activity, which may be cultured in MacConkey selective culture medium, under aerobic conditions at 37° C. For example, species belonging to the genus: Escherichia coli, Cytrobacter freundii, Klebsiella. The two gene regions containing the target sequences of our Coliforms detection are the GAL coding lacZ and the GUS coding uidA.
In our invention procedure the pathogen EC E. coli germ designation includes the bacteria that can be genetically specified as Escherichia coli taxonomical species and, furthermore, that has the SHG+ ability of shigatoxin enterotoxin production, and which may be cultured in selective culture medium TTC 2,3,5-triphenyltetrazolium chloride broth, under aerobic conditions at 30° C. and 37° C. For example species belonging to the Escherichia coli EPEC, ETEC, EIEC serotypes. The two gene regions containing the target sequences of our E. coli detection are the EC 16S RNA coding ec16s-rna and the shigatoxin coding stx1.
In our invention procedure the designation PA Pseudomonas aeruginosa germs causing potential illness includes the bacteria that can be genetically specified as Pseudomonas aeruginosa taxonomical species and, furthermore, that are ITLP+ capable of Iturin toxic lipopeptide production, and which may be cultured in BHI Brain Heart Infusion general culture medium, under aerobic conditions at 37° C. The two gene regions containing the target sequences of our Pseudomonas aeruginosa detection are the PA16S RNA coding pa16s-rna and the Iturin lipopeptide coding it.
In our invention procedure the designation of the pathogen EF Enterococcus faecalis germs includes the bacteria that can be genetically specified as the Enterococcus faecalis taxonomical species and, furthermore, that have the EEP+ determinant for enhanced expression of pheromones ability producing haemolysin/bacteriocin, and which may be cultured in Azide Dextrose selective culture medium, under aerobic conditions at 37° C. The two gene regions containing the target sequences of our E. faecalis detection are the EF16S RNA coding ef16s-rna and the EEP coding eep.
In our invention procedure the designation of the pathogen CP Clostridium perfringens germs includes the bacteria that can be genetically specified as the Clostridium perfringens taxonomical species and, furthermore, that have the CPAB+ clostridium perfringens alpha-beta toxin production ability, and which may be cultured in Schaedler selective culture medium, under anaerobic conditions at 37° C. The two gene regions containing the target sequences of our Clostridium perfringens detection are the CP16S RNA coding cp16s-rna and the CPAB coding cpAB.
In our invention procedure the designation of the pathogen Salmonella enterica germs includes the bacteria that can be genetically specified as the Salmonella enterica taxonomical species and, furthermore, has the VERO+ capability to produce verocytotoxin, and which may be cultured in Selenite-cysteine broth selective culture medium, under aerobic conditions at 37° C. Examples include the Salmonella typhi, S. paratyphi, S. typhimurium. The two gene regions containing the target sequences of our Salmonella enterica detection are the SE16S RNA coding se16s-rna and the enterotoxin coding ver.
In our invention procedure the designation of Staphylococcus aureus germs, which potentially causes illness, includes the bacteria that can be genetically specified as the Staphylococcus aureus taxonomical species and, furthermore, has the COAG+ capability to produce coagulase enzyme, and which may be cultured in Giolitti-Cantoni broth selective culture medium, under aerobic conditions at 37° C. The two gene regions containing the target sequences of our Staphylococcus aureus detection are the SA16S RNA coding sa16s-rna and the COAG coding coa.
In our invention procedure the designation of the enteric pathogen Campylobacter jejuni germs includes the bacteria that can be genetically specified as the Campylobacter jejuni and coli taxonomical species and, furthermore, has the ETB+ capability to produce enterotoxinB, and which may be cultured in Azide Dextrose broth selective culture medium, under aerobic conditions at 37° C. The two gene regions containing the target sequences of our Campylobacter jejuni detection are the CJ16S RNA coding cj16s-rna and the ETB coding cetB.
In our invention procedure the designation of the pathogen Listeria monocytogenes germs includes the bacteria that can be genetically specified as the Listeria monocytogenes taxonomical species and, furthermore, has the HLY+ capability to produce haemolysin (listeriolysin), and which may be cultured in Fraser broth selective culture medium, under aerobic conditions at 37° C. The two gene regions containing the target sequences of our Listeria monocytogenes detection are the LM16S RNA coding lm16s-rna and the HLY coding hly.
In our invention procedure the designation of the enteric pathogen Shigella germs includes the bacteria that can be genetically specified as the Shigella flexneri taxonomical species and, furthermore, has the STs+ capability to produce shigatoxin A, and which may be cultured in Selenite cysteine broth selective culture medium, under aerobic conditions at 37° C. The two gene regions containing the target sequences of our Shigella flexneri detection are the SF16S RNA coding sf16 s-rna and the STs A coding stx2 .
In our invention procedure the designation of the nosocomial pathogen MRSA germs includes the bacteria that can be genetically specified as the Staphylococcus aureus taxonomical species and, furthermore, has the PBP2a+ capability to produce the penicillin binding protein 2a responsible for methicillin resistance, and which after the selective isolation of Staphylococcus aureus may be cultured in ORSA broth selective culture medium, under aerobic conditions at 37° C. The two gene regions containing the target sequences of our methicillin resistant Staphylococcus aureus detection are the SA16S RNA coding sa16s-rna and the PBP2a coding mecA. In our invention procedure the designation of the pathogen Legionella germs includes the bacteria that can be genetically specified as the Legionella pneumophila taxonomical species and, furthermore, has the MIP+ capability to produce the macrophage infectivity factor, and which after the selective isolation of the Legionella content of the samples may be cultured in Legionella charcoal—BHI Brain Heart Infusion selective culture medium, under aerobic conditions at 37° C. The two gene regions containing the target sequences of our Legionella pneumophila detection are the LP16S RNA coding lp16s-rna and the macrophage IP coding coding mip.
In our invention procedure the designation of the pathogen Mycobacterium germs includes the bacteria causing tuberculosis that can be genetically specified as the Mycobacterium tuberculosis taxonomical species and, furthermore, contains the bacteria carrying Mycobacterium tuberculosis complex IS6110 insertion element-infectivity factor and, which may be cultured in MGIT Mycobacterium growth indicator broth selective culture medium, under aerobic conditions at 37° C. Examples include the M. bovis, M. bovis BCG, M. smegmatis, M. avium, M. tuberculosis species. The two gene regions containing the target sequences of our Mycobacterium tuberculosis complex detection are the MTB16S RNA coding mtb16s-rna and the MTC IS6110 coding is6110.
   The recognitions of our invention procedure according to the above are summarised as follows.

The false positive results of the PCR methods used to date in microbial diagnostics, which aroused uncertainty in the users in connection with the applicability of the method, may also be traced back, among other reasons, to the primer annealing to target faults recognised by us. We base our PCR detections on the primers annealing to new genetic target sequences different to those used to date, through this we provide new genetic specificity for our system (see FIG. 1). With all this we emphasise that with the oligonucleotide primer and fluorescent oligo probe sequences defining the specificity of the determination of bacterial germ count according to our procedure and of the KITs realising our procedure in practice, we detect operating genes, because we detect the targeted structural genes together with the adjacent upstream regulatory promoter-operator region sequences. The characteristics of the oligonucleotides planned for the new genetic target sequences, i.e. the forward and reverse primer pairs and of the oligo probes have been summarised in table 3 (for the details see the table 3 description).

Regarding our embodiment examples for the calibrated determination of HPC22, HPC37 and Coliforms bacterial germ counts, the 5′-3′ orientation sequences of the forward and reverse primer pairs and of the oligo probes planned by us are appended in table 4 and listed from SEQ ID NO 1 to SEQ ID NO 12 in the sequence listing.

In order to increase detection reliability, we build our duplex technique on the joint detection (see FIG. 1: target gene 1, target gene 2) of the evolutionarily conserved (e.g. ribosomal RNA or 16S RNA) genes and the genes responsible for pathogenicity (e.g. Listeria haemolysin, Campylobacter enterotoxin, Salmonella verocytotoxin, Staphylococcus coagulase). Beside this with the detection of the adjacent upstream regulatory promoter-operator region sequences we can also make conclusions on functionality. Following the description detailing the figures we have listed the new genetic target sequences including the templates to the PCR probe elements planned by us, i.e. templates to forward and reverse primers, internal hydrolysis probes for the duplex dual colour detection detailed in our invention specification. Accordingly, the template sequences from SEQ ID NO 13 to SEQ ID NO 124 are appended, and the international genetic database references are shown.

The technical differences between the detecting instruments of real-time PCR technology and the specificity characteristic of the given instrument has to date not made the application of universal oligonucleotide labelling technology possible. The reason for this is that in the majority of cases the specific, pronounced emission maximum given by certain fluorophores cannot be detected on real-time PCR instruments of other manufacturers, only on dedicated devices. The detection channels of the dedicated instruments evaluate the fluorescent signal emitted by the channel-specific dye in a small range of 10-15 nm, and they are unable to effectively detect the signals of similar but not identical fluorophores with close emission maxima.

We find the elaboration of a MasterMix reaction mixture necessary with which the detection of the signal generated by fluorescent-labelled oligo probes is made possible on as many real-time PCR measurement instruments as possible. The additive substances of the 2× concentrated MasterMix optimised by us, favourably DMSO dimethyl sulfoxide, FAME fatty acid methyl ester C8-C10 fraction, ANS amino-naphtalenyl-sulfonic acid, and the effective dilutions of their favourably 10-15 μg/ml stock solutions (see table 1) widen the fluorescence spectrum of the dyes used. This Fluorescence Shift, extension towards the red spectrum, is simple, reliable from the aspect of quantitative evaluation, and due to this the emission spectra of the individual real-time PCR dyes give an emission maximum in a wider range. In other words the intensity peak of the emitted light may be detected, instead of in the narrow z10 nm wavelength range, in the wider wavelength range of 20-35 nm. Due to this our detection system ensures a reaction that can be repeated with the appropriate degree of precision and with excellent fluorescence characteristics on most of the PCR platforms that are available on the market (Roche, ABI, BioRad, Corbett, Stratagene, etc.).

The dual colour fluorescence labelling system of our duplex technique is iso-fluorescein-amino-methyl+iso-tetramethyl-rhodamin, and iso-carboxyl-dichloro-dimethoxyfluorescein+iso-tetramethyl-rhodamine, which dyes are components of the PCR reaction medium, the 2× concentrated MasterMix mixture according to our procedure (see table 1).

Our reactions specific for new genetic target sequences and our Fluorescence Shift technique make it possible to set up the unique profile of the PCR programs to be used in the detection of the individual indicator bacteria. This is a new element that is a time-temperature profile set up as a program for each indicator bacterium examined, exclusively specific for the PCR reactions given by us. Accordingly, the nucleic acid based molecular diagnostic determination of bacterial germ counts according to our invention procedure is favourably carried out according to the PCR profiles shown in tables 5-18.

Time-saving PCR, and especially real-time PCR is very advantageous as compared to the culturing technique in the faster performance of public health and clinical hygiene testing. Until now an attested, validated standard that expressed the result in an appropriate measurement range was lacking in quantitative PCR practices used in the determination of bacterial germ counts. For this reason, in the practices to date, quantitative PCR measurement results could not really be compared with the measurement results of the culturing procedures used in public health and clinical hygiene practices.

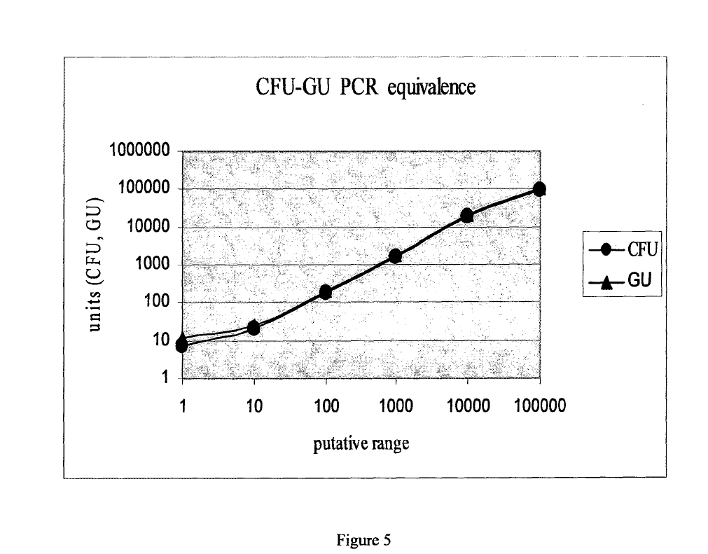

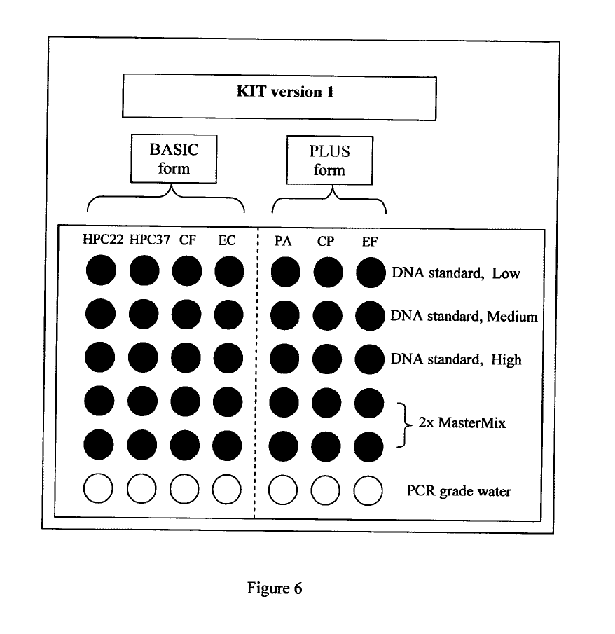

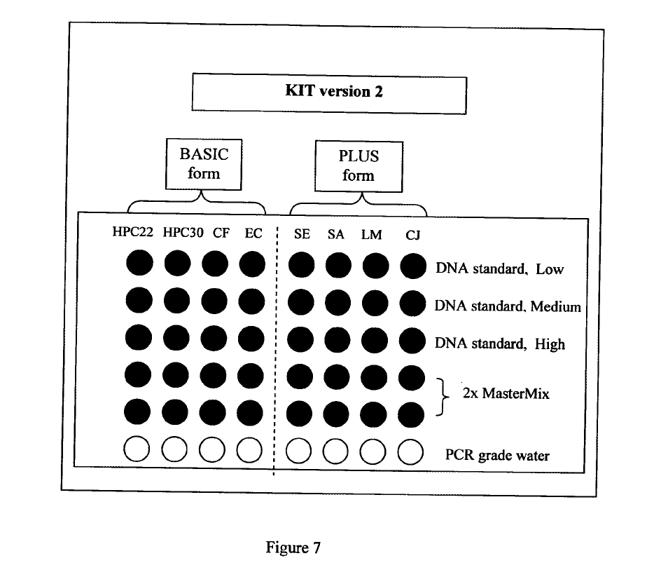

We have elaborated a form of quantitative determination with the same measurement range as the traditional standard procedures, which provides the same measurement units, which makes new, nucleic acid based calibration possible that is not absolute but related to the given standard sample unit, volume and mass unit. Following 16 h of selective enrichment culturing in the standard culturing medium we isolate DNA from CFU germs of the unknown sample and of the microbe identical Reference Material calibrator sample in standard volume-units and standard mass-units. In the following real-time PCR reaction we determine the calibration points according to the standard with the microbe identical sample calibrator GU genome unit equivalent DNA amount, and with the help of this we characterise the bacterial germ count of the unknown sample with the GU genome unit equivalent DNA amount. The traditional reference sample volume is 1 ml or 1 g, for these standard sample units the three calibration points (low-medium-high) are the 10 CFU or 100 CFU or 1000 CFU microbe identical total germ count found in the sample, furthermore for the 100 ml or 100 g standard sample unit the three calibration points (low-medium-high) are the 1 CFU or 10 CFU or 100 CFU microbe identical total germ count found in the sample. For the checking of our solution, the absolute positive control is the GU genome unit equivalent amount of DNA isolated from CFU germs of microbe identical CRM Certified Reference Material standard sample unit following 16 h of selective enrichment culturing. In the range given with our calibration measurements we characterise the CFU germ number with the GU genome unit equivalent DNA amount, as is illustrated by FIG. 5 (for details see the description of the figure). According to our three-point calibration related to the standard sample units detailed above, in our invention procedure and in the KITs realising the procedure the microbe identical three calibration points are indicated with the designations DNA standard, Low—DNA standard, Medium—DNA standard, High (see FIGS. 6 and 7, for details see the descriptions of the figures).

On the basis of the above it is clearly advantageous for real-time PCR technology to be introduced as a reliable, validated and quantitative instrumental measurement procedure in microbiology diagnostics practices (clinics, public healthcare, water and food product hygiene).

The subject of the invention then relates to nucleic acid-based molecular diagnostic determination of bacterial germ counts during which we detect evolutionarily conserved genes and genes coding for characteristic pathogenicity markers favourably microbial enzyme, toxin, special resistance. It is characteristic of our nucleic acid-based diagnostic detection that with our real-time PCR method at the same time as detecting the presence of the structural genes we also check the possibility of their functionality by detecting the 5′ upstream regulatory promoter-operator sequences. In a further advantageous embodiment of the invention we use standard developed and validated by us in the determination of bacterial germ counts in accordance with our procedure to evaluate the results, with which we measure the real-time PCR results with GU genome unit equivalent DNA amount calibrated to the CFU germ number of standard sample units.

In accordance with the above when realising our nucleic acid-based molecular diagnostic procedure as one element of PCR probes we use the structural gene 5′ end region and as the other element we favourably use the adjacent upstream regulatory promoter-operator region sequences.

As instrumental measurement in our nucleic acid-based diagnostic procedure we favourably use the real-time PCR method. That method of realisation of our invention procedure is very favourable where we use duplex amplification during the real-time PCR analysis for the simultaneous detection of the two kinds of marker, i.e. the evolutionarily conserved taxonomic marker and the characteristic pathogenicity marker as genetic targets in the reaction space. In a further advantageous embodiment of our invention procedure in our duplex amplification system we work with dual dye fluorescence labelling that can be detected in two different wavelength ranges. To this dual colour technique we optimised the PCR reaction mixture with DMSO dimethyl sulfoxide, FAME fatty acid methyl ester C8-C10 fraction, ANS amino-naphtalenyl-sulfonic acid additives, as a result of which the reaction elaborated by us may be run on any real-time PCR platform, in other words universally.

With respect to the above the solution is very favourable in which we use isofluorescein-amino-methyl+iso-tetramethyl-rhodamine, and iso-carboxyl-dichloro-dimethoxyfluorescein+iso-tetramethyl-rhodamine dyes in the dual colour fluorescent labelling in the PCR reaction mixture optimised with the aforementioned additives resulting in Fluorescence Shift widening of the two detection wavelength ranges.

We base the calibrated determination according to our procedure of bacterial germ counts favourably on single copy gene sequences in the genome, like those coding for characteristic pathogenicity markers listed before.

The subject of the invention relates to a procedure for nucleic acid-based molecular diagnostic determination of HPC total Heterotrophic Plate Count/Total bacterial germ count during which the presence of core16s-rna and gapdh structural genes is shown in samples with real-time PCR method, in the course of which as one element of PCR probes we use the 5′ end region of the core16s-rna and gapdh structural genes, and as the other element we use the adjacent upstream regulatory promoter-operator region sequences. For the detection of the presence of structural genes mentioned above the oligonucleotide forward primer, reverse primer and fluorescent labelled probe planned by us comply with SEQ ID NO 1, SEQ ID NO 2, SEQ ID NO 3 and SEQ ID NO 4, SEQ ID NO 5, SEQ ID NO 6 sequences (see table 4). For the detection of the presence of structural genes mentioned above the templates to hybridization annealing of SEQ ID NO 1, SEQ ID NO 2, SEQ ID NO 3 and SEQ ID NO 4, SEQ ID NO 5, SEQ ID NO 6 planned by us are included in SEQ ID NO 13 and SEQ ID NO 17, favourably complying with SEQ ID NO 14, SEQ ID NO 15, SEQ ID NO 16 and SEQ ID NO 18, SEQ ID NO 19, SEQ ID NO 20 sequences (see sequence listing).

The subject of the invention further relates to the procedure for the nucleic acid-based molecular diagnostic determination of Coliforms germ counts during which the presence of lacZ and uidA structural genes is shown in samples with real-time PCR method, in the course of which as one element of PCR probes we use the 5′ end region of the lacZ and uidA structural genes, and as the other element we use the adjacent upstream regulatory promoter-operator region sequences. For the detection of the presence of structural genes mentioned above the oligonucleotide forward primer, reverse primer and fluorescent labelled probe planned by us comply with SEQ ID NO 7, SEQ ID NO 8, SEQ ID NO 9, and SEQ ID NO 10, SEQ ID NO 11, SEQ ID NO 12 sequences (see table 4). For the detection of the presence of structural genes mentioned above the templates to hybridization annealing of SEQ ID NO 7, SEQ ID NO 8, SEQ ID NO 9, and SEQ ID NO 10, SEQ ID NO 11, SEQ ID NO 12 planned by us are included in SEQ ID NO 21 and SEQ ID NO 25, favourably complying with SEQ ID NO 22, SEQ ID NO 23, SEQ ID NO 24 and SEQ ID NO 26, SEQ ID NO 27, SEQ ID NO 28 sequences (see sequence listing).

The subject of the invention also relates to the procedure for the nucleic acid-based molecular diagnostic determination of Escherichia coli germ counts during which the presence of ec16s-rna and stx1 structural genes is shown in samples with real-time PCR method, in the course of which as one element of PCR probes we use the 5′ end region of the ec16s-rna and stx1 structural genes, and as the other element we use the adjacent upstream regulatory promoter-operator region sequences. For the detection of the presence of structural genes mentioned above the templates to hybridization annealing of forward primer, reverse primer and fluorescent labelled probe planned by us are included in SEQ ID NO 29 and SEQ ID NO 33 favourably complying with SEQ ID NO 30, SEQ ID NO 31, SEQ ID NO 32 and SEQ ID NO 34, SEQ ID NO 35, SEQ ID NO 36 sequences (see sequence listing).

The subject of the invention furthermore relates to the procedure for the nucleic acid-based molecular diagnostic determination of Pseudomonas aeruginosa germ counts during which the presence of pa16s-rna and it structural genes is shown in samples with real-time PCR method, in the course of which as one element of PCR probes we use the 5′ end region of the pa16s-rna and it structural genes, and as the other element we use the adjacent upstream regulatory promoter-operator region sequences. For the detection of the presence of structural genes mentioned above the templates to hybridization annealing of forward primer, reverse primer and fluorescent labelled probe planned by us are included in SEQ ID NO 37 and SEQ ID NO 41 favourably complying with SEQ ID NO 38, SEQ ID NO 39, SEQ ID NO 40 and SEQ ID NO 42, SEQ ID NO 43, SEQ ID NO 44 sequences (see sequence listing).

The subject of the invention also relates to the procedure for the nucleic acid-based molecular diagnostic determination of Enterococcus faecalis germ counts during which the presence of ef16s-rna and eep structural genes is shown in samples with real-time PCR method, in the course of which as one element of PCR probes we use the 5′ end region the ef16s-rna and eep structural genes, and as the other element we use the adjacent upstream regulatory promoter-operator region sequences. For the detection of the presence of structural genes mentioned above the templates to hybridization annealing of forward primer, reverse primer and fluorescent labelled probe planned by us are included in SEQ ID NO 45 and SEQ ID NO 49 favourably complying with SEQ ID NO 46, SEQ ID NO 47, SEQ ID NO 48 and SEQ ID NO 50, SEQ ID NO 51, SEQ ID NO 52 sequences (see sequence listing).

The subject of the invention also relates to the procedure for the nucleic acid-based molecular diagnostic determination of Clostridium perfringens germ counts during which the presence of cp16s-rna and cpAB structural genes is shown in samples with real-time PCR method, in the course of which as one element of PCR probes we use the 5′ end region of the cp16s-rna and cpAB structural genes, and as the other element we use the adjacent upstream regulatory promoter-operator region sequences. For the detection of the presence of structural genes mentioned above the templates to hybridization annealing of forward primer, reverse primer and fluorescent labelled probe planned by us are included in SEQ ID NO 53 and SEQ ID NO 57 favourably complying with SEQ ID NO 54, SEQ ID NO 55, SEQ ID NO 56 and SEQ ID NO 58, SEQ ID NO 59, SEQ ID NO 60 sequences (see sequence listing).

The subject of the invention relates to the procedure for the nucleic acid-based molecular diagnostic determination of Salmonella enterica germ counts during which the presence of se16s-rna and ver structural genes is shown in samples with real-time PCR method, in the course of which as one element of PCR probes we use the 5′ end region of the se16s-rna and ver structural genes, and as the other element we use the adjacent upstream regulatory promoter-operator region sequences. For the detection of the presence of structural genes mentioned above the templates to hybridization annealing of forward primer, reverse primer and fluorescent labelled probe planned by us are included in SEQ ID NO 61 and SEQ ID NO 65 favourably complying with SEQ ID NO 62, SEQ ID NO 63, SEQ ID NO 64 and SEQ ID NO 66, SEQ ID NO 67, SEQ ID NO 68 sequences (see sequence listing).

The subject of the invention relates to the procedure for the nucleic acid-based molecular diagnostic determination of Staphylococcus aureus germ counts during which the presence of sa16s-rna and coa structural genes is shown in samples with real-time PCR method, in the course of which as one element of PCR probes we use the 5′ end region of the sa16s-rna and coa structural genes, and as the other element we use the adjacent upstream regulatory promoter-operator region sequences. For the detection of the presence of structural genes mentioned above the templates to hybridization annealing of forward primer, reverse primer and fluorescent labelled probe planned by us are included in SEQ ID NO 69 and SEQ ID NO 73 favourably complying with SEQ ID NO 70, SEQ ID NO 71, SEQ ID NO 72 and SEQ ID NO 74, SEQ ID NO 75, SEQ ID NO 76 sequences (see sequence listing).

The subject of the invention relates to the procedure for the nucleic acid-based molecular diagnostic determination of Campylobacter jejuni/coli germ counts during which the presence of cj16s-rna and cetB structural genes is shown in samples with real-time PCR method, in the course of which as one element of PCR probes we use the 5′ end region of the cj 16s-rna and cetB structural genes, and as the other element we use the adjacent upstream regulatory promoter-operator region sequences. For the detection of the presence of structural genes mentioned above the templates to hybridization annealing of forward primer, reverse primer and fluorescent labelled probe planned by us are included in SEQ ID NO 77 and SEQ ID NO 81 favourably complying with SEQ ID NO 78, SEQ ID NO 79, SEQ ID NO 80 and SEQ ID NO 82, SEQ ID NO 83, SEQ ID NO 84 sequences (see sequence listing).

The subject of the invention relates to the procedure for the nucleic acid-based molecular diagnostic determination of Listeria monocytogenes germ counts during which the presence of 1M16s-rna and hly structural genes is shown in samples with real-time PCR method, in the course of which as one element of PCR probes we use the 5′ end region of the 1M16s-rna and hly structural genes, and as the other element we use the adjacent upstream regulatory promoter-operator region sequences. For the detection of the presence of structural genes mentioned above the templates to hybridization annealing of forward primer, reverse primer and fluorescent labelled probe planned by us are included in SEQ ID NO 85 and SEQ ID NO 89 favourably complying with SEQ ID NO 86, SEQ ID NO 87, SEQ ID NO 88 and SEQ ID NO 90, SEQ ID NO 91, SEQ ID NO 92 sequences (see sequence listing).

The subject of the invention relates to the procedure for the nucleic acid-based molecular diagnostic determination of Shigella flexneri germ counts during which the presence of sf16s-rna and stx2 structural genes is shown in samples with real-time PCR method, in the course of which as one element of PCR probes we use the 5′ end region of the sf16s-rna and stx2 structural genes, and as the other element we use the adjacent upstream regulatory promoter-operator region sequences. For the detection of the presence of structural genes mentioned above the templates to hybridization annealing of forward primer, reverse primer and fluorescent labelled probe planned by us are included in SEQ ID NO 93 and SEQ ID NO 97 favourably complying with SEQ ID NO 94, SEQ ID NO 95, SEQ ID NO 96 and SEQ ID NO 98, SEQ ID NO 99, SEQ ID NO 100 sequences (see sequence listing).

The subject of the invention relates to the procedure for the nucleic acid-based molecular diagnostic determination of MRSA Methicillin rezisztens Staphylococcus aureus germ counts during which the presence of sa16s-rna and mecA structural genes is shown in samples with real-time PCR method, in the course of which as one element of PCR probes we use the 5′ end region of the sa16s-rna and mecA structural genes, and as the other element we use the adjacent upstream regulatory promoter-operator region sequences. For the detection of the presence of structural genes mentioned above the templates to hybridization annealing of forward primer, reverse primer and fluorescent labelled probe planned by us are included in SEQ ID NO 101 and SEQ ID NO 105 favourably complying with SEQ ID NO 102, SEQ ID NO 103, SEQ ID NO 104 and SEQ ID NO 106, SEQ ID NO 107, SEQ ID NO 108 sequences (see sequence listing).

The subject of the invention relates to the procedure for the nucleic acid-based molecular diagnostic determination of Legionella pneumophila germ counts during which the presence of lp16s-rna and mip structural genes is shown in samples with real-time PCR method, in the course of which as one element of PCR probes we use the 5′ end region of the lp16s-rna and mip structural genes, and as the other element we use the adjacent upstream regulatory promoter-operator region sequences. For the detection of the presence of structural genes mentioned above the templates to hybridization annealing of forward primer, reverse primer and fluorescent labelled probe planned by us are included in SEQ ID NO 109 and SEQ ID NO 113 favourably complying with SEQ ID NO 110, SEQ ID NO 111, SEQ ID NO 112 and SEQ ID NO 114, SEQ ID NO 115, SEQ ID NO 116 sequences (see sequence listing).

The subject of the invention relates to the procedure for the nucleic acid-based molecular diagnostic determination of Mycobacterium tuberculosis germ counts during which the presence of mtb16s-rna and is6110 structural genes is shown in samples with real-time PCR method, in the course of which as one element of PCR probes we use the 5′ end region of the mtb16s-rna and is6110 structural genes, and as the other element we use the adjacent upstream regulatory promoter-operator region sequences. For the detection of the presence of structural genes mentioned above the templates to hybridization annealing of forward primer, reverse primer and fluorescent labelled probe planned by us are included in SEQ ID NO 117 and SEQ ID NO 121 favourably complying with SEQ ID NO 118, SEQ ID NO 119, SEQ ID NO 120 and SEQ ID NO 122, SEQ ID NO 123, SEQ ID NO 124 sequences (see sequence listing).

A further subject of the invention is the KITs serving the practical realisation of the nucleic acid based molecular diagnostic determination of bacterial germ counts (see FIG. 6 for KIT version 1, FIG. 7 for KIT version 2).

In the following we give a detailed description to the figures and the appended tables in order of appearance.

FIG. 1. In the detection of target gene 1 (evolutionarily conserved gene coding taxonomic marker) and target gene 2 (gene coding pathogenicity marker, favourably enzyme, toxin, special resistance) sequences in the figure we have marked the recent technologies relying on the internal sequences of the structural genes with a black arrow. The double-line arrow indicates the essence of duplex, dual colour detection according to our procedure, in which as one element of PCR probes we use the structural gene 5′ end region and as the other element we use the adjacent upstream regulatory promoter-operator sequences. Through this the fluorescent labelled hydrolysis probes according to our procedure inform us of such amplified nucleotide chains that contain the structural genes along with the adjacent upstream regulatory promoter-operator sequences.

Table 1. Favourable composition of the 2× concentrated MasterMix (2× MasterMix) for the microbe specific duplex, dual colour fluorescent real-time PCR reaction. In the individual bacterial detections the sequences of the planned oligonucleotide set i.e. the forward primer, reverse primer, probe in the reaction-optimised 2× concentrated MasterMix (2× MasterMix) medium varies in accordance with the microbial template sequences (see description). NTPs=nucleotide triphosphates, BSA=bovine serum albumin, TRIS-HCl=Tris(hydroxymethyl)aminomethane—hidrochloride buffer, MgCl₂=magnesium chloride, DMSO=dimethyl sulfoxide, FAME=fatty acid methyl ester C8-C10 fraction, ANS=amino naphtalene sulfonic acid, NaCl=sodium chloride, KCl=potassium chloride

Table 2. The mixing of 2× MasterMix according to table 1, with the PCR grade distilled water and the DNA isolated from the test sample for the duplex, dual colour microbe specific real-time PCR reaction, to a final volume of 20 μl. Table 3. In the quantitative determination of bacterial germs according to our procedure, the characteristics of the target gene specific forward-reverse primer pairs and fluorescent labelled probes (see description). The amount of GC guanine-cytosine base pairs in primers determine the temperature stability of primer-template hybridization. The high GC content results in a higher Tm melting point in the microbe specific duplex, dual colour real-time PCR program (see later on). We indicate the length of the primers in by base pairs.

Table 4. Sequences of primers and probes planned by us for the duplex, dual colour, calibrated determination of the HPC22, HPC37 and Coliforms germ counts as described in the embodiment examples. Symbol “n” stands for optional nucleotide in the oligonucleotide sequence.

Tables 5-18. In the table summary of the microbe specific duplex, dual colour real-time PCR programs we have shown the temperature, time, ramping rate and acquisition mode/analysis characteristics of the cyclic denaturation-annealing-extension phases following starting denaturation, and, furthermore, the holding conditions following the cycles. The 530 nm and 560 fluorescence maxima of the duplex, dual colour reactions optimised originally for the Roche Light Cycler® 2.0 device as a result of our Fluorescence Shift may be reliably detected on other PCR devices as well.

The microbe specific duplex, dual colour real-time PCR programs set up by us for the determination of bacterial germ counts listed in the specification are detailed below.

Tables 5-6. Heterotrophic Plate Count (HPC), Coliforms (CF)

Tables 7-8. E. coli (EC), Pseudomonas aeruginosa (PA)

Tables 9-10. Enterococcus faecalis (EF), Clostridium perfringens (CP)

Tables 11-12. Salmonella enterica (SE), Staphylococcus aureus (SA)

Tables 13-14. Campylobacter jejuni/coli (CJ), Listeria monocytogenes (LM)

Tables 15-16. Shigella flexneri (SF), Methicillin resistant Staphylococcus aureus (MRSA)

Tables 17-18. Legionella pneumophila (LP), Mycobacterium Tuberculosis Complex (MTC)

Table 19. Nucleic acid based molecular diagnostic determination of bacterial germ counts of test samples with specific real-time PCR reaction, for the practical illustration of embodiment example 2. In the HPC22 and HPC37 example the maximum 200 ng/ml DNA content per PCR reaction isolated from the microbe identical 10¹ CFU-10² CFU-10³ CFU germs of the three standard, i.e. st1 low, st2, medium, st3 high calibration samples serve for the determination of the GU genome unit equivalent DNA amounts of the three calibration points (low-medium-high) of the 1 ml reference sample volume (see description). U . . . =isolated DNA content of the unknown sample. The absolute reference of our procedure is the microbe identical CRM Certified Reference Material-DNA (see description) at a concentration of 100× diluted, maximum 200 ng/ml.

Table 20. Nucleic acid based molecular diagnostic determination of bacterial germ counts of test samples with specific real-time PCR reaction, for the practical illustration of embodiment example 3. In the Coliforms example the maximum 200 ng/ml DNA content per PCR reaction isolated from the microbe identical 10⁰ CFU-10¹ CFU-10² CFU germs of the three standard, i.e. st1 low, st2, medium, st3 high calibration samples serve for the determination of the GU genome unit equivalent DNA amounts of the three calibration points (low-medium-high) of the 100 ml reference sample volume (see description). U . . . =isolated DNA content of the unknown sample. The absolute reference of our procedure is the microbe identical CRM Certified Reference Material-DNA (see description) at a concentration of 50× diluted, maximum 200 ng/ml.

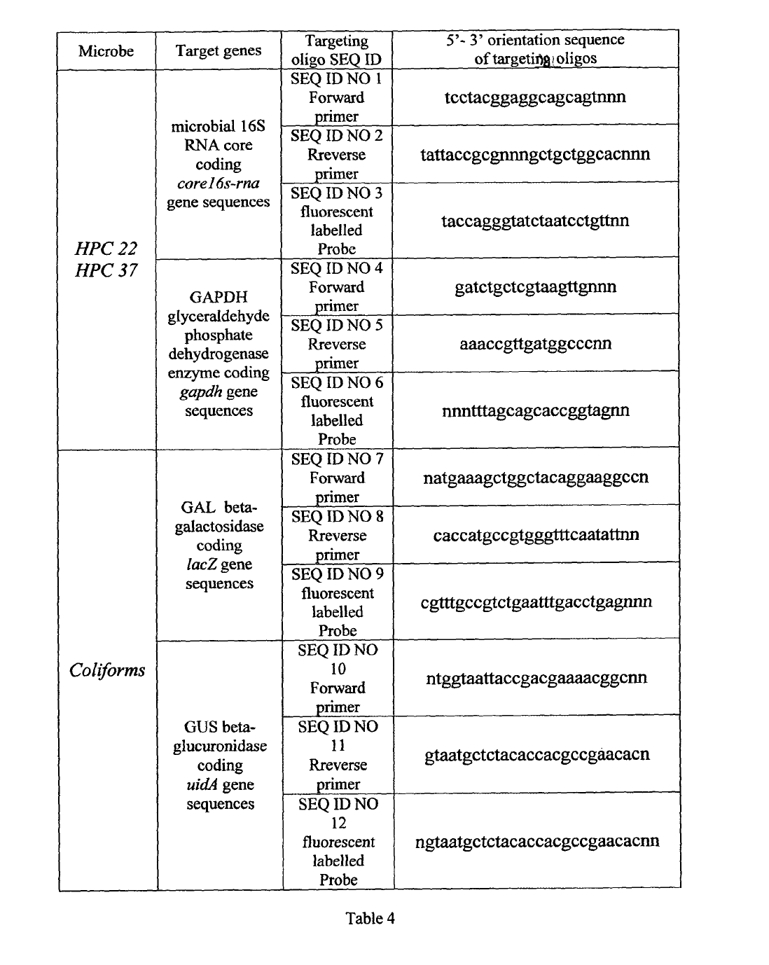

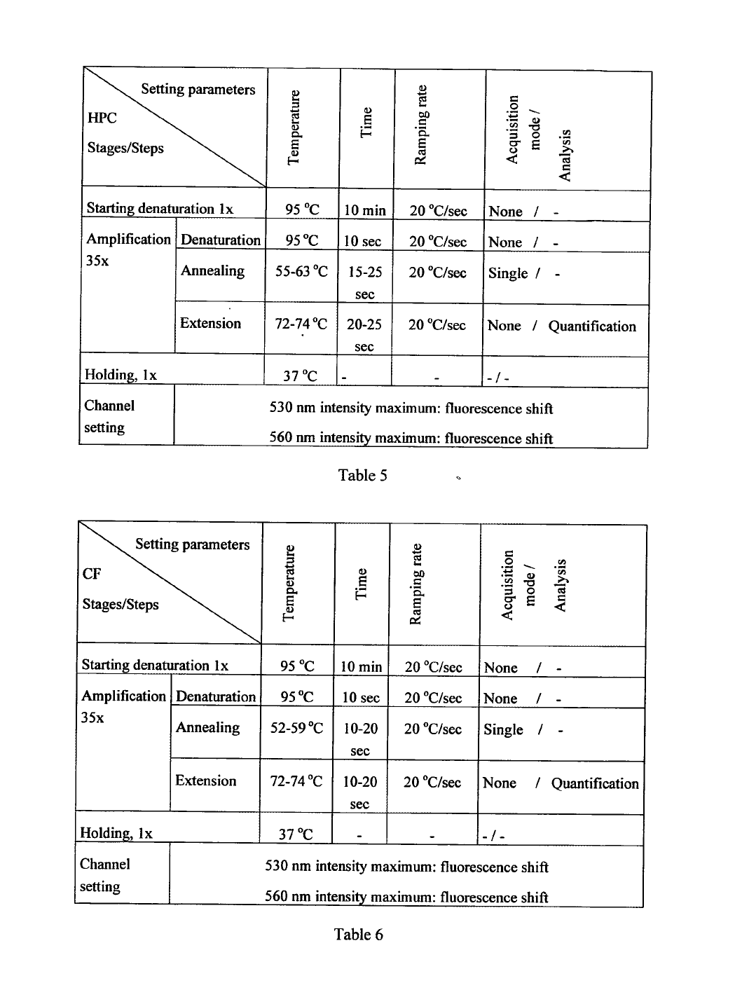

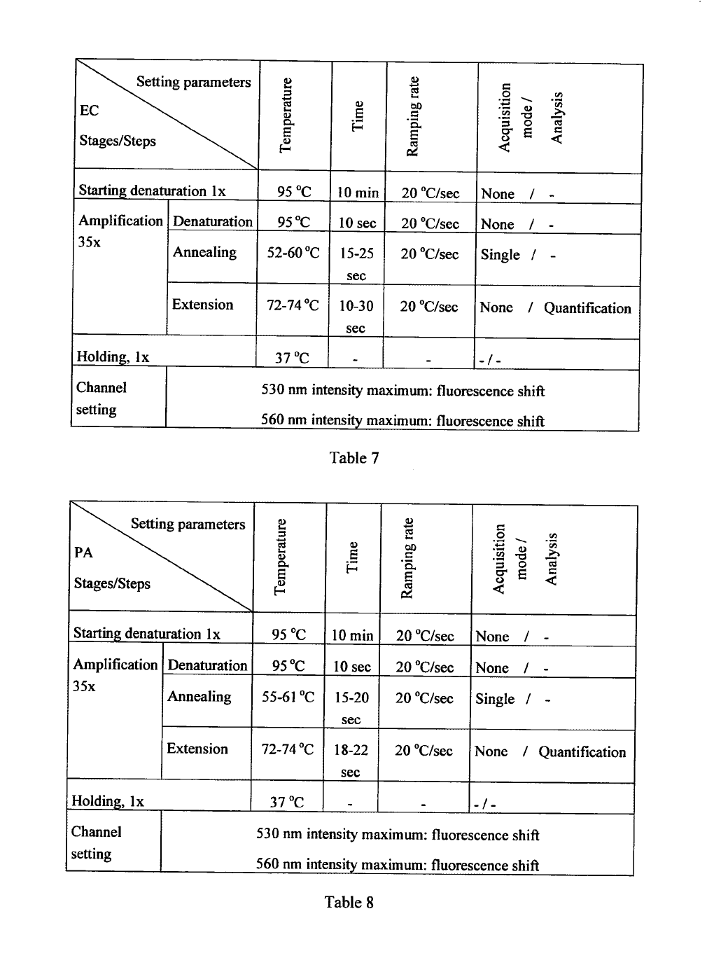

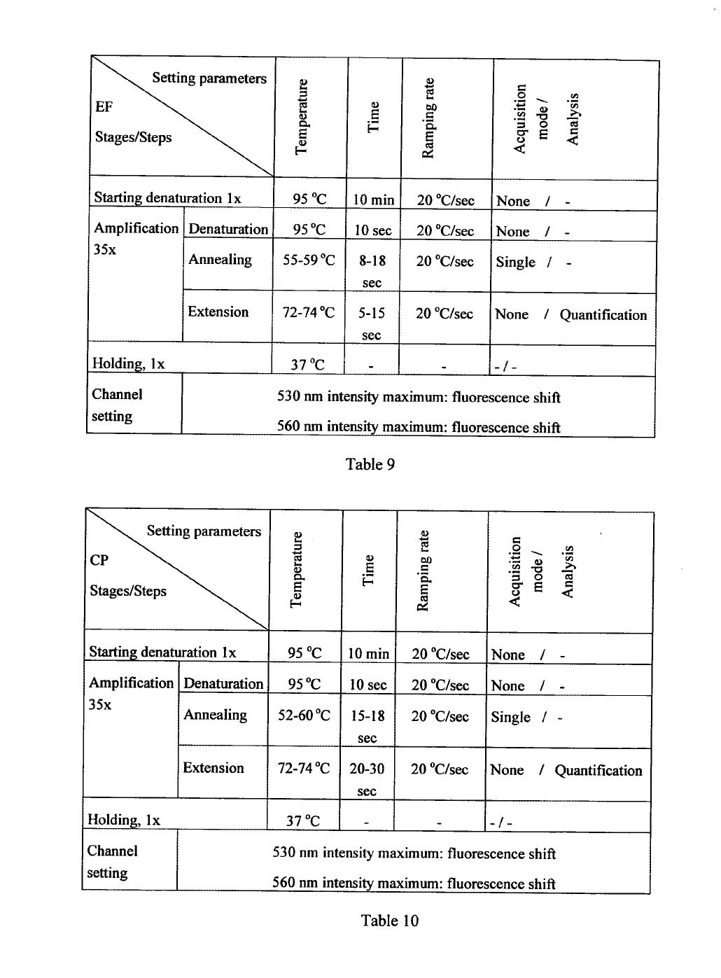

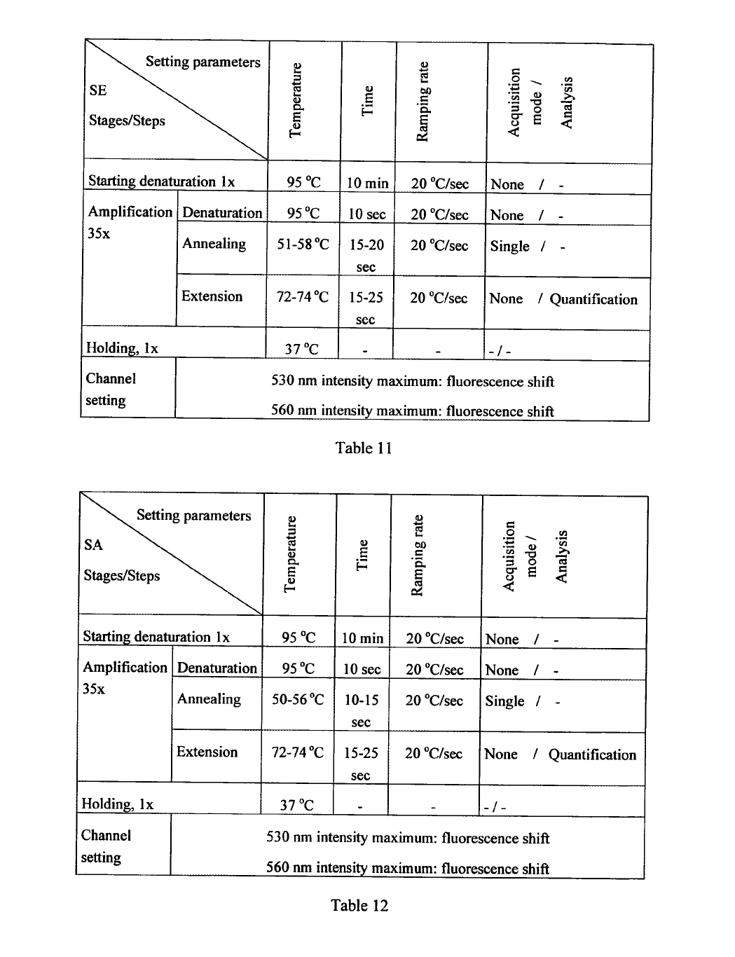

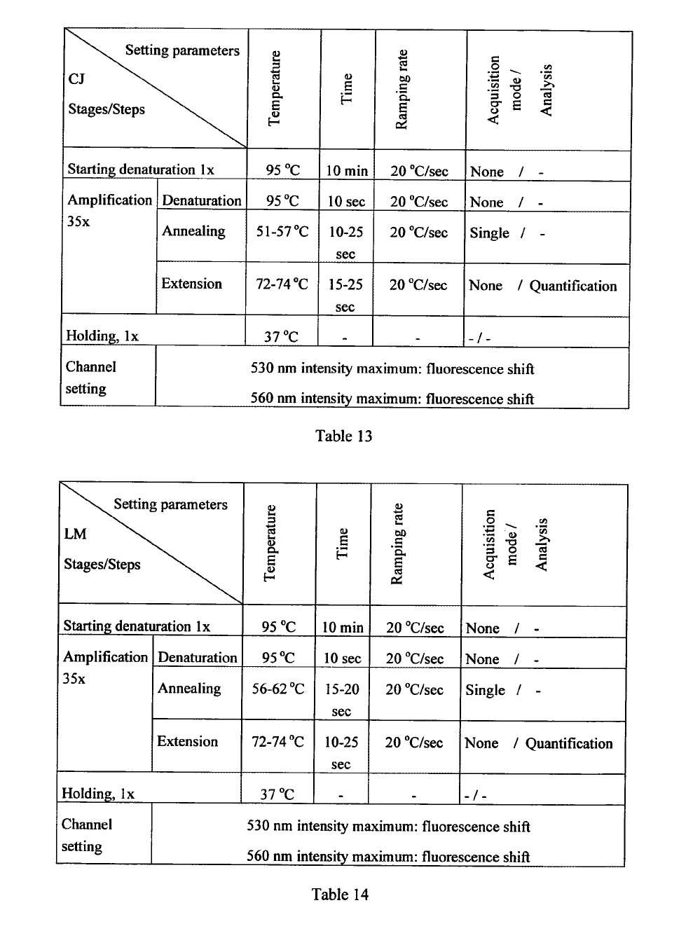

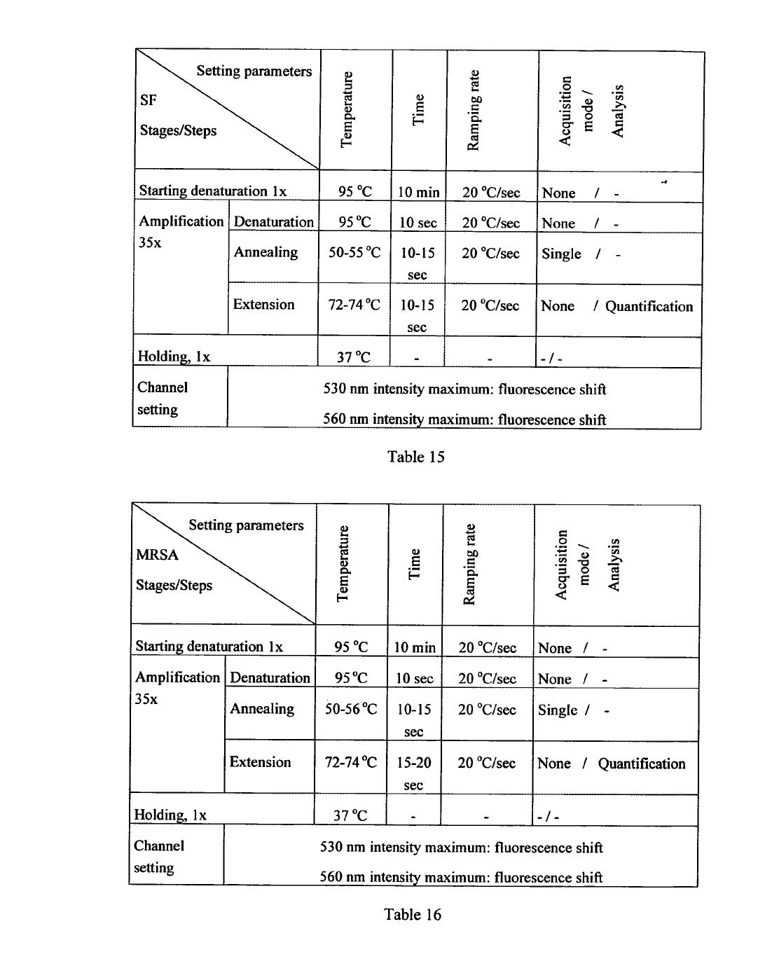

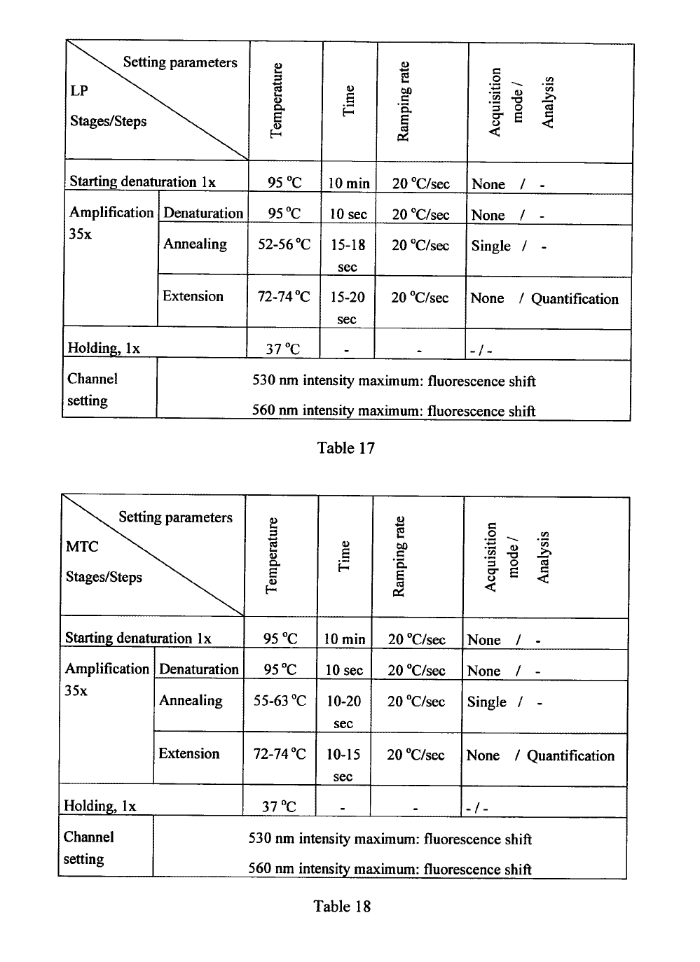

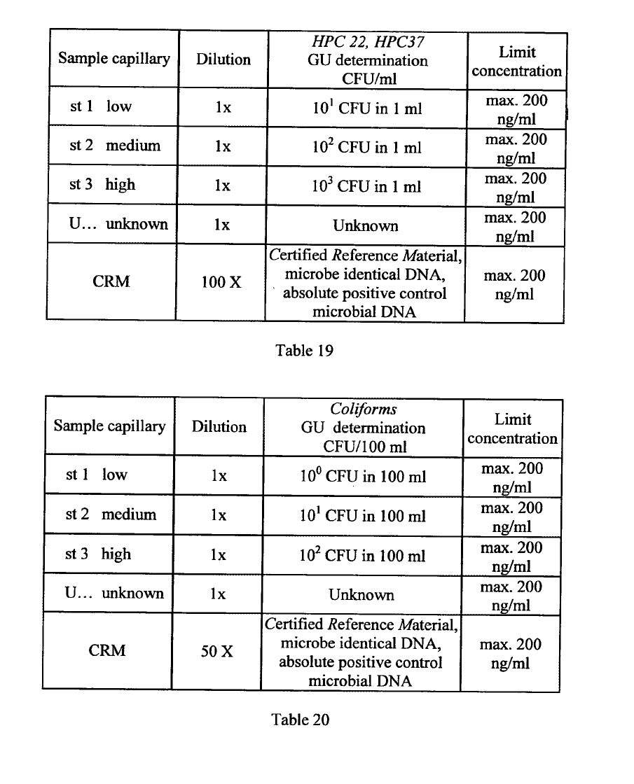

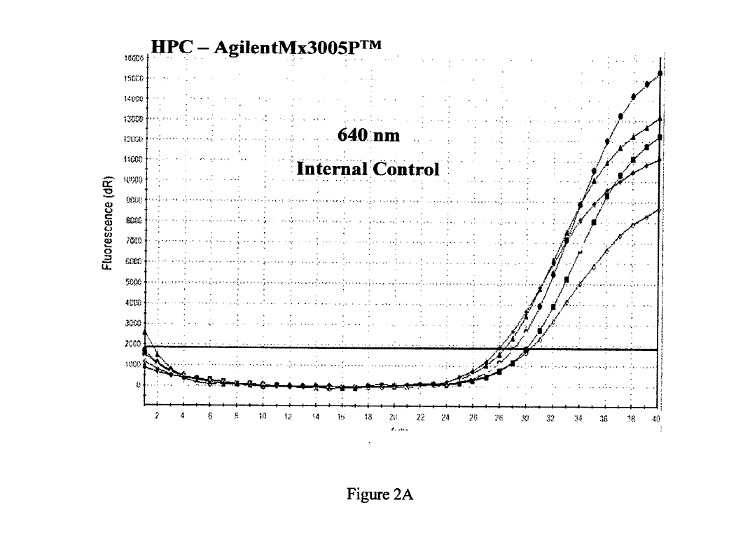

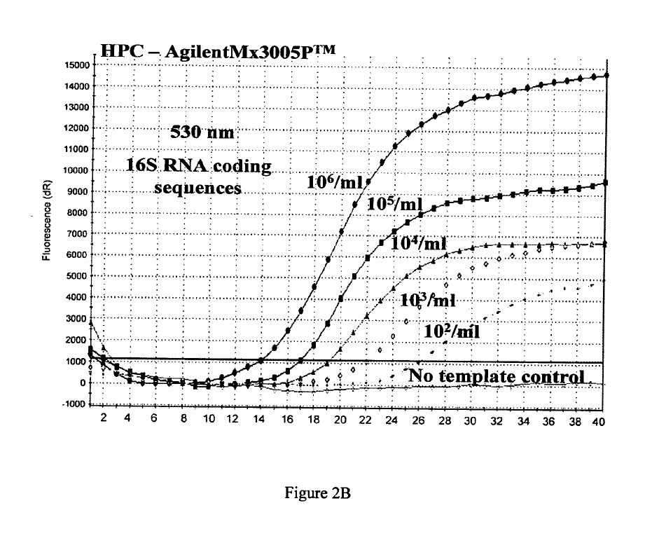

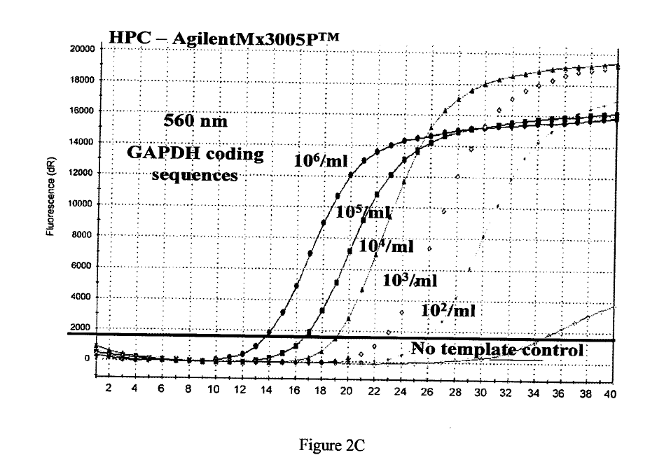

FIG. 2. Detection of HPC22-37 Gram positive and Gram negative bacteria according to our duplex, dual color procedure. FIG. 2A (detection at 640 nm)=real-time PCR internal control reaction with template sequences coding for bacterial lectin. The internal control shows the technical reliability of the reaction performed. FIG. 2B (detection at 530 nm)=detection of HPC22 and HPC37 by the new genetic target sequences in core16s-rna coding for 16S RNA core. Reaction curves marked with 10⁶/ml-10⁵/ml-10⁴/ml-10³/ml-10²/ml show the inverse relationship between CFU starting concentration and the Cp cycle number (see description). FIG. 2C (detection at 560 nm)=detection of HPC22 and HPC37 by the new target sequences in gapdh coding for bacterial GAPDH glyceraldehyde phosphate dehydrogenase enzyme. Reaction curves marked with 106/ml-105/ml-104/ml-103/ml-102/ml show the inverse relationship between CFU starting concentration and the Cp cycle number (see description).

y axis is dR relative fluorescence plotted against x axis PCR reaction cycle number.

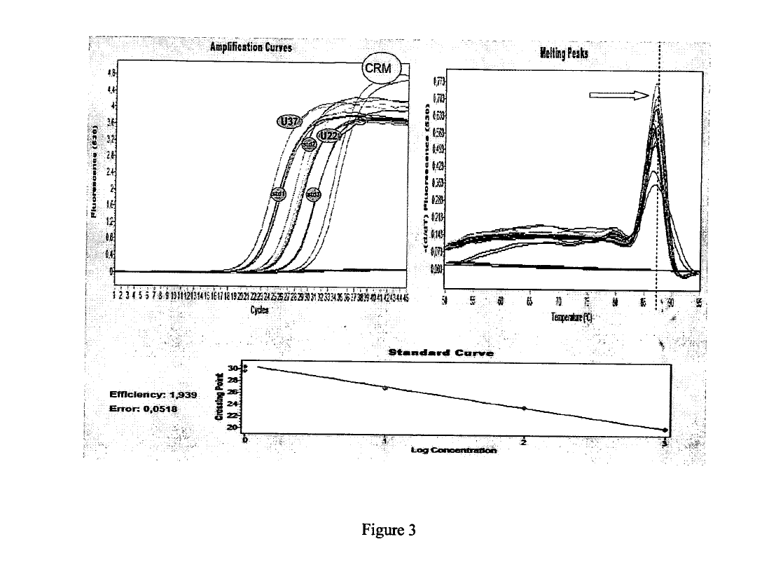

FIG. 3. Determination of HPC22 and HPC37 germ counts according to our procedure (see embodiment example 2).

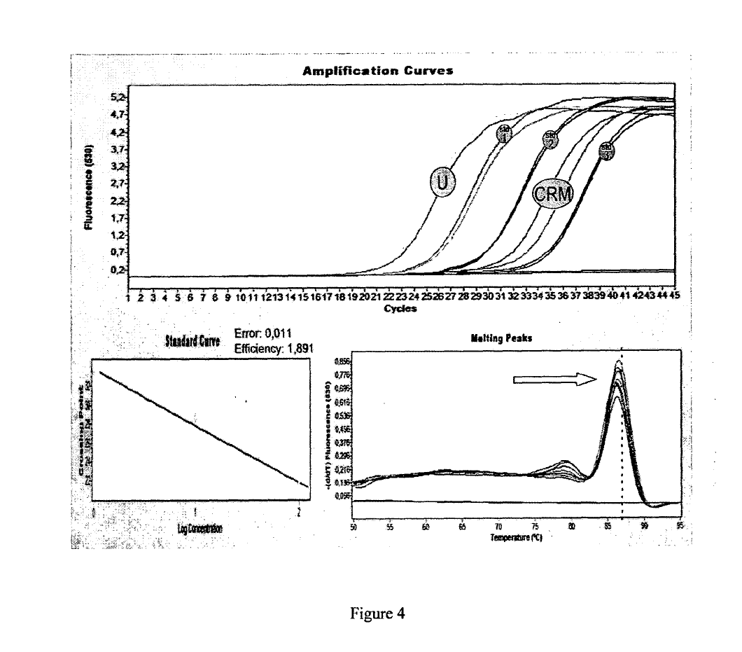

FIG. 4. Determination of Coliforms germ counts according to our procedure (see embodiment example 3).

FIG. 5. The CFU-GU equivalence presentation for the determination of bacterial germ counts according to our invention procedure in the Legionella pneumophila example, with the help of the macrophage infectivity factor coding mip gene. The x-axis shows the increasing series of the reference sample units, i.e. the reference sample volumes and sample masses (see description). The y-axis shows the GU genome unit equivalent DNA amount isolated from the CFU germs of the reference sample units according to the x-axis. It can be easily seen that the CFU and the GU values cover each other well at every single of the reference sample units, and there is only a very slight deviation at great dilution (see y-axis values in the 1-10 range of the x-axis).

FIG. 6. KIT version 1 for the comprehensive water testing system unified for the most frequently tested parameters of drinking water bacteriology and general bacteriology microbial detections. As it can be seen in the figure the reagent columns marked with the microbe to be detected in series one under the other contain the standards (Low, Medium, High) making three-point calibration possible serving the detection of the bacterium, the specific 2× MasterMix and the PCR grade water required for the dilution of the PCR reagents. The KIT version 1 does not contain the primer required for the performance of the individual reactions.

Testing parameters in the KIT version 1:

| | |
|---|---|
| Basic form | HPC22 (Heterotrophic Plate Count, 22° C.) |
| | HPC37 (Heterotrophic Plate Count, 37° C.) |
| | CF (Coliforms) |
| | EC (Escherichia coli) |
| Plus form | PA (Pseudomonas aeruginosa) |
| | CP (Clostridium perfringens) |
| | EF (Enterococcus faecalis) |

FIG. 7. KIT version 2 for the comprehensive food industry testing system unified for the most frequently tested parameters of food product hygiene and general bacteriology microbial detections. As it can be seen in the figure the reagent columns marked with the microbe to be detected in series one under the other contain the standards (Low, Medium, High) making three-point calibration possible serving the detection of the bacterium, the specific 2× MasterMix and the PCR grade water required for the dilution of the PCR reagents. The KIT version 2 does not contain the primer required for the performance of the individual reactions.

Testing parameters in the KIT version 2:

| | |
|---|---|
| Basic form | HPC22 (Heterotrophic Plate Count, 22° C.) |
| | HPC30 (Heterotrophic Plate Count, 30° C.) |
| | CF (Coliforms) |
| | EC (Escherichia coli) |
| Plus form | SE (Salmonella enterica) |
| | SA (Staphylococcus aureus) |
| | LM (Listeria monocytogenes) |
| | CJ (Campylobacter jejuni) |

### EMBODIMENT EXAMPLES

Nucleic acid-based molecular diagnostic determination of bacterial germ counts with real-time PCR method. The reaction optimized for capillary real-time PCR device (Roche LightCycler® 2.0), may also be run on other platforms (for example, see FIG. 2). Before the procedure according to the invention is realised we isolate the bacterial DNA content of the sample, we check its amount with conventional UV spectrophotometry (λ=260 nm). One OD unit conforms to 50 μg/ml DNA concentration.

We characterise the purity of the isolated DNA with the ratio of the optical density values measured at the λ=260 nm and λ=280 nm wavelengths. For PCR tests the range OD₂₆₀/OD₂₈₀=1.4-1.8 is appropriate.

### Example 1

The detection of bacterial germs with the duplex, dual colour real-time PCR procedure
a) From the planned-synthesized-lyophilized primer pairs and internal probes and with the addition of the amount of PCR-grade water stated on the accompanying synthesis sheet we make 100 pmol/μl stock solution. Our hydrolysis probe (internal probe) is marked with the light-sensitive fluorescent dyes iso-carboxy-dichloro-dimethoxy-fluorescein and iso-fluorescein-amino-methyl. The emission maxima of the two dyes are 560 nm and 530 nm. During the work processes in order to fully achieve the Fluorescence Shift we pay special attention to making sure that it is only illuminated by low intensity light for as short a time as possible.
b) We then pipette a 10 μl amount of the 100 pmol/μl stock solution made as above into a sterile Eppendorf tube and dilute it further into working solution with a concentration of 10 pmol/μl with the addition of 90 μl PCR grade water. The preparation of this latter working solution is essential to avoid the contamination of the stock solution. We then thoroughly homogenise the primer-probe working solution prepared in this way with a pipette.
c) We then prepare the 2× MasterMix with the composition given in table 1, in such a way that the primer-probe working solutions prepared in accordance with the previous points a-b are added to it subsequently, in the final concentrations according to table 1.
   The compulsory features of the operational steps a-c are the following:
      the stock solution is to be stored frozen.
      when making the stock solution the laboratory protocols relating to PCR procedures are to be strictly followed (EN-ISO 20838:2006, GLP).
      work to be strictly carried out in clean room environment (ISO 209, FS 209, BS5295, ISO 14644-1:1999), at a safety level of min. BSL2 (Biosafety Level 2).
d) Table 2 presents the duplex, dual colour fluorescence labelled specific real-time PCR reaction set up, for a final volume of 20 μl per reaction. According to table 2 add together the template DNA, the 2× MasterMix solution prepared according to table 1 and the PCR grade water. Take care to ensure that the final concentration of the template DNA does not exceed the critical 200 ng value.
e) Run the reaction according to the specific PCR program given for the bacterial germ to be detected. The technical details of the microbe specific PCR programs are contained in tables 4-17.
   The obligatory features of the operation steps d-e are the following:
      add together the reaction substances at a temperature of between +2 and +8° C.
      when adding together the reaction substances the standard laboratory protocols relating to PCR procedures are to be strictly followed (EN-ISO 20838:2006, GLP)
      work to be strictly carried out in clean room environment (ISO 209, FS 209, BS5295, ISO 14644-1:1999), at a safety level of min. BSL2 (Biosafety Level 2)

### Example 2

The calibrated determination of bacterial germ counts in the HPC22-HPC37 example. The new genetic targets of our duplex dual-color detection are illustrated in FIG. 1. In table 4 we present SEQ ID NO 1, SEQ ID NO 2, SEQ ID NO 3 and SEQ ID NO 4, SEQ ID NO 5, SEQ ID NO 6 sequences of annealing primers and probes planned by us for the PCR reactions. Further, we append from SEQ ID NO 13 to SEQ ID NO 20 the complementary template sequences included in the PCR reactions. The sequence listing is shown with the genetic database source.

Following 16 h of preliminary enrichment (culture medium at the descriptive listing of the indicator bacteria) we carry out the calibrated detection of the bacterial germ count of the test sample in the dynamic measurement range 10 CFU/ml-100 CFU/ml-1000 CFU/ml according to table 19. Accordingly, we set up the specific PCR reaction volumes for our measurement from that listed in table 1 and table 2 referred to in embodiment example 1 so that their individual isolated template DNA content originates from the following samples: dynamic measurement range high CFU standard, dynamic measurement range medium CFU standard, dynamic measurement range low CFU standard, U unknown sample. The DNA amount in the individual reaction volumes should be a maximum of 200 ng/ml. In HPC22-HPC37 examples the real-time PCR program should be run according to the parameters in table 5. For the absolute positive control reaction we use DNA reference isolated from microbe identical HPC-CRM (see text) cells, in a maximum concentration of 200 ng/ml. The upper left-hand insert of FIG. 3 shows the kinetics of the PCR reactions of the HPC22-HPC37 standard (std1, std2, std3), the U (U22, U37) unknown and the absolute reference positive control HPC-CRM samples, with the Cp values created with the measuring software of the device used. Remember, the Cp value is the cycle number when the intensity of fluorescence indicating the presence of the searched-for template, exceeds the background level, when the amplification reaction enters the exponential phase. The specificity of the reactions may be checked with Tm melting point analysis, this can be seen in the upper right-hand insert. It is conspicuous that the melting point of the PCR products coincides, the reaction is specific.

In the lower insert the relationship, inverse proportionality of the Cp values (y axis) and the CFU log values (x axis) equivalent to the calibrating standard series genome unit equivalent DNA amount can be seen. With the help of this calibration algorithm the Cp value of the unknown sample generated by the measurement may be converted into CFU/ml data.

### Example 3

The calibrated detection of bacterial germ counts in the Coliforms example. The new genetic targets of our duplex dual-color detection are illustrated in FIG. 1. In table 4 we present SEQ ID NO 7, SEQ ID NO 8, SEQ ID NO 9 and SEQ ID NO 10, SEQ ID NO 11, SEQ ID NO 12 sequences of annealing primers and probes planned by us for the PCR reactions. Further, we append from SEQ ID NO 21 to SEQ ID NO 28 the complementary template sequences included in the PCR reactions. The sequence listing is shown with the genetic database source.

Following 16 h of preliminary enrichment (culture medium at the descriptive listing of the indicator bacteria) we carry out the calibrated detection of the bacterial germ count of the test sample in the dynamic measurement range 1 CFU/100 ml-10 CFU/100 ml-100 CFU/100 ml according to table 20. Accordingly, we set up the specific PCR reaction volumes for our measurement from that listed in table 1 and table 2 referred to in embodiment example 1 so that their individual isolated template DNA content originates from the following samples: dynamic measurement range high CFU standard, dynamic measurement range medium CFU standard, dynamic measurement range low CFU standard, U unknown sample. The DNA amount in the individual reaction volumes should be a maximum of 200 ng/ml. In Coliforms examples the real-time PCR program should be run according to the parameters in table 6. For the absolute positive control reaction we use DNA reference isolated from microbe identical Coliforms-CRM (see text) cells, in a maximum concentration of 200 ng/ml.

The upper insert of FIG. 4 shows the kinetics of the PCR reactions of the Coliforms standard (std1, std2, std3), the U unknown and the absolute reference positive control Coliforms-CRM samples, with the Cp values created with the measuring software of the device used. Remember, the Cp value is the cycle number when the intensity of fluorescence indicating the presence of the searched-for template, exceeds the background level, when the amplification reaction enters the exponential phase. The specificity of the reactions may be checked with Tm melting point analysis, this can be seen in the lower right-hand insert. It is conspicuous that the melting point of the PCR products coincides, the reaction is specific.

In the lower left-hand insert the relationship, inverse proportionality of the Cp values (y axis) and the CFU log values (x axis) equivalent to the calibrating standard series genome unit equivalent DNA amount can be seen. With the help of this calibration algorithm the Cp value of the unknown sample generate by the measurement may be converted into CFU/100 ml data.

The measurements according to the examples shown may also be performed with other technology than capillary real-time PCR technology, like, for example, with microplate real-time PCR technology. In the latter case due to the differing fluorescence characteristics and detection technology before starting the measurement it is recommended that the detection system be calibrated and colour compensation performed according to the program given in the manufacturer's instructions of the PCR device. It is to be emphasised that all the reagents of our procedure we produced as our own development.

Two commercial embodiment examples of the KITs according to the invention may be seen in FIGS. 6 and 7.

The technical advantages of our invention are good measurement technology parameters, speed and sensitivity, which characteristics we certify with the following data.
Good measurement technology parameters
   Low CV % (Coefficient of variation): 10.5-6.9%
   Specificity: 89-94%
   Repeatability: max. 95%
   Dynamic linear measurement range-1:1-10-100 CFU/100 ml (100 g)
   Dynamic linear measurement range-2: 10-100-1000 CFU/ml (g)
   LOD(Limit of Detection)-1: 1 CFU/100 ml +/−5% (absolute)
   LOD(Limit of Detection)-2: 1 CFU/ml +/−10% (absolute)
   LOQ(Limit of Quantitation)-1:1 CFU/100 ml
   LOQ(Limit of Quantitation)-2: 1 CFU/ml
Speed: a result may be obtained in 18 hours as opposed to the traditional 72 hours
The instrument-sensitivity of the reaction according to our procedure is indicated by that it may be used on practically the most real-time PCR platforms.

The economic advantages of our invention are speed and precision, which characteristics we certify with the following data.
Speed—The measurement takes place in a short time, 16 hours selective pre-culturing and 2 hours PCR, through this it is possible to implement fast hygienic interventions for a company operating a quality control system, water works or food product plant. So the discharges, control tests and the return of the product does not lead to losses of time. For example, a meat product does not have to stand in a warehouse for 3 days, for example, until the traditional test period has passed. Large drinking water discharge deposits may become unnecessary planned due to the long testing periods in a water plant to store 3 days of discharge in several stages. Instead the test results that are available quickly make it possible to immediately discharge the treated water or treat it once again.
Precision—Instrumental measurement for microbiology that can be more easily made independent from human error, as opposed to the culturing procedures that many times accept empirical and human factors, and errors. For the laboratory user it is a fast, precise method with which wage costs may be saved.

The areas of application of our invention are drinking water and wastewater bacteriology, water works control laboratories, food product testing stations, food product industry control laboratories, general bacteriology, workplace hygiene, as well as occupational and public healthcare.

1-41. (canceled)

42. A procedure usable for nucleic acid-based molecular diagnostic determination of bacterial germ counts, wherein:
evolutionarily conserved genes and genes coding for characteristic pathogenicity markers are detected,
for said detection real-time PCR duplex amplification with dual colour fluorescence method is used with the help of priming oligonucleotides and fluorescent labelled hydrolysis probes,
in said duplex amplification, as one element of PCR probes the structural gene 5′ end region is used,
in said duplex amplification, as the other element of PCR probes the adjacent upstream regulatory promoter-operator region sequences are used,
using said PCR probe elements, the presence of the structural gene is shown along with the adjacent upstream regulatory promoter-operator region sequences,
the use of said PCR probe elements makes simultaneously to check the functional nature of the structural gene,
the two fluorescent dyes for said detection are iso-fluorescein-amino-methyl+iso-tetramethyl-rhodamine and iso-carboxyl-dichloro-dimethoxyfluorescein+iso-tetramethyl-rhodamine,
said fluorescent dyes are in reading wavelength range widening PCR buffer solutions, favourably with DMSO, FAME fraction, ANS additives,
the result of the said real-time PCR amplification is measured with a genome unit equivalent DNA amount—GU-,
said genome unit equivalent DNA amount—GU—is calibrated to the bacterial germ number—CFU—of sample units equivalent to standard procedures,
said calibration to the bacterial germ number is based on single copy gene sequences in the genome, like those coding for characteristic pathogenicity markers,
for said nucleic acid-based molecular diagnostic determination of bacterial germ counts the calibration standard is the DNA content of the microbe identical Reference Material,
with said microbe identical Reference Material the germ counts of the traditional three standard calibration points, the 1 CFU/100 ml-10 CFU/100 ml-100 CFU/100 ml and the 1 CFU/100 g-10 CFU/100 g-100 CFU/100 g, furthermore the 10 CFU/1 ml-100 CFU/1 ml-1000 CFU/1 ml and the 10 CFU/1 g-100 CFU/1 g-1000 CFU/1 g is expressed with the genome unit equivalent-GU-amount.

43. The procedure according to claim 42, usable for nucleic acid-based molecular diagnostic determination of bacterial germ counts, wherein the presence of the:
core16s-rna and gapdh structural genes,
lacZ and uidA structural genes,
ec16s-rna and stx1 structural genes,
pa16s-rna and it structural genes,
ef16s-rna and eep structural genes,
cp16s-rna and cpAB structural genes,
se16s-rna and ver structural genes,
sa16s-rna and coa structural genes,
cj16s-rna and cetB structural genes,
lm16s-rna and hly structural genes,
sf16s-rna and stx2 structural genes,
sal16s-rna and mecA structural genes,
lp16s-rna and mip structural genes,
mtb16s-rna and is6110 structural genes
along with the adjacent upstream regulatory promoter-operator region sequences is shown in water and/or food samples for the said determination of:
Total heterotrophic plate count/Total bacterial germ count HPC
Coliforms
Escherichia coli
Pseudomonas aeruginosa
Enterococcus faecalis
Clostridium perfringens
Salmonella enterica
Staphylococcus aureus
Campylobacter jejuni/coli
Listeria monocytogenes
Shigella flexneri
Methicillin resistant Staphylococcus aureus, MRSA
Legionella pneumophila
Mycobacterium tuberculosis bacterial germ counts, respectively.

44. The procedure according to claim 42, usable for nucleic acid-based molecular diagnostic determination of Total heterotrophic plate count/Total bacterial germ count HPC, and Coliforms bacterial germ counts in water and food samples, wherein:
the said priming oligonucleotides forward primer, reverse primer, the fluorescent labelled probe comply with SEQ ID NO 1 to 3; SEQ ID NO 4 to 6 and SEQ ID NO 7 to 9; SEQ ID NO 10 to 12 sequences, respectively,
the templates to said forward primer, reverse primer, fluorescent labelled probe comply with SEQ ID NO 14, SEQ ID NO 15, SEQ ID NO 16; SEQ ID NO 18, SEQ ID NO 19, SEQ ID NO 20 and SEQ ID NO 22, SEQ ID NO 23, SEQ ID NO 24; SEQ ID NO 26, SEQ ID NO 27, SEQ ID NO 28 sequences, respectively.

45. The procedure according to claim 43, usable for nucleic acid-based molecular diagnostic determination of Escherichia coli bacterial germ counts in water and food samples, Pseudomonas aeruginosa, Enterococcus faecalis, Clostridium perfringens bacterial germ counts in water samples, wherein the templates to said forward primer, reverse primer, fluorescent labelled probe comply with:
SEQ ID NO 30 to 32; SEQ ID NO 34 to 36 sequences,
SEQ ID NO 38 to 40; SEQ ID NO 42 to 44 sequences,
SEQ ID NO 46 to 48; SEQ ID NO 50 to 52 sequences,
SEQ ID NO 54 to 56; SEQ ID NO 58 to 60 sequences, respectively.

46. The procedure according to claim 43, usable for nucleic acid-based molecular diagnostic determination of Salmonella enterica, Staphylococcus aureus, Campylobacter jejuni/coli, Listeria monocytogenes bacterial germ counts in food samples, wherein the templates to said forward primer, reverse primer, fluorescent labelled probe comply with:
SEQ ID NO 62 to 64; SEQ ID NO 66 to 68 sequences,
SEQ ID NO 70 to 72; SEQ ID NO 74 to 76 sequences,
SEQ ID NO 78 to 80; SEQ ID NO 82 to 84 sequences,
SEQ ID NO 86 to 88; SEQ ID NO 90 to 92 sequences, respectively.

47. The procedure according to claim 43, usable for nucleic acid-based molecular diagnostic determination of Shigella flexneri, Methicillin resistant Staphylococcus aureus MRSA, Legionella pneumophila, Mycobacterium tuberculosis bacterial germ counts in samples, wherein the templates to said forward primer, reverse primer, fluorescent labelled probe comply with:
SEQ ID NO 94 to 96; SEQ ID NO 98 to 100 sequences,
SEQ ID NO 102 to 104; SEQ ID NO 106 to 108 sequences,
SEQ ID NO 110 to 112; SEQ ID NO 114 to 116 sequences,
SEQ ID NO 118 to 120; SEQ ID NO 122 to 124 sequences, respectively.

48. KIT for the realisation of said procedure of claim 42, for the nucleic acid-based molecular diagnostic determination of bacterial germ counts; said KIT in Basic Forms and Plus Forms containing DNA standards, specific 2× MasterMix, PCR grade water, for the determination of Pseudomonas aeruginosa and/or Enterococcus faecalis and/or Clostridium perfringens from water samples, Salmonella enterica, Staphylococcus aureus and/or Campylobacter jejuni/coli and/or Listeria monocytogenes from food samples, Total heterotrophic plate count/Total bacterial germ count HPC and/or Coliforms and/or Escherichia coli from water and food samples.

49. A set of annealing primers and probes usable in a process according to claim 42 for determining bacterial germ counts of HPC22-HPC37 and/or of Coliforms comprising one or more polynucleotides with a nucleotide sequence selected from SEQ ID NO 1 to SEQ ID NO 6 and/or SEQ ID NO 7 to SEQ ID NO 12.

50. A set of primers and probes according to claim 49, wherein
SEQ ID NO 1 is a forward primer,
SEQ ID NO 2 is a reverse primer,
SEQ ID NO 3 is a fluorescent labelled probe,
SEQ ID NO 4 is a forward primer,
SEQ ID NO 5 is a reverse primer,
SEQ ID NO 6 is a fluorescent labelled probe,
SEQ ID NO 7 is a forward primer,
SEQ ID NO 8 is a reverse primer,
SEQ ID NO 9 is a fluorescent labelled probe,
SEQ ID NO 10 is a forward primer,
SEQ ID NO 11 is a reverse primer, and
SEQ ID NO 12 is a fluorescent labelled probe.

51. A set of primers and probes according to claim 49, wherein the set comprises primers and probes with the nucleotide sequences of
SEQ ID NOs 1 to 12,
SEQ ID NOs 1 to 6, or
SEQ ID NOs 7 to 12, respectively.

52. A set of complementary templates for hybridizing annealing primers and probes usable in a process according to claim 42 for determining bacterial germ counts of HPC22-HPC37 and/or of Coliforms comprising one or more polynucleotides with a nucleotide sequence selected from SEQ ID NO 14 to SEQ ID NO 16 and SEQ ID NO 18 to SEQ ID NO 20, and/or SEQ ID NO 22 to SEQ ID NO 24 and SEQ ID NO 26 to SEQ ID NO 28.

53. A set of complementary templates according to claim 52, wherein
SEQ ID NO 14 is a template to a forward primer,
SEQ ID NO 15 is a template to a reverse primer,
SEQ ID NO 16 is a template to a fluorescent labelled probe,
SEQ ID NO 18 is a template to a forward primer,
SEQ ID NO 19 is a template to a reverse primer,
SEQ ID NO 20 is a template to a fluorescent labelled probe,
SEQ ID NO 22 is a template to a forward primer,
SEQ ID NO 23 is a template to a reverse primer,
SEQ ID NO 24 is a template to a fluorescent labelled probe,
SEQ ID NO 26 is a template to a forward primer,
SEQ ID NO 27 is a template to a reverse primer, and
SEQ ID NO 28 is a template to a fluorescent labelled probe.

54. A set of complementary templates according to claim 52, wherein the set comprises templates for primers and probes with the nucleotide sequences of
SEQ ID NO 1 to SEQ ID NO 6, or
SEQ ID NO 7 to SEQ ID NO 12, respectively.

55. A set of complementary templates for hybridizing annealing primers and probes usable in a process according to claim 42 for determining bacterial germ counts of Escherichia coli, Pseudomonas aeruginosa, Enterococcus faecalis, Clostridium perfringens, Salmonella enterica, Staphylococcus aureus, Campylobacter jejuni/coli, Listeria monocytogenes, Shigella flexneri, Methicillin resistant Staphylococcus aureus, Legionella pneumophila and/or of Mycobacterium tuberculosis comprising one or more polynucleotides with a nucleotide sequence selected from
SEQ ID NO 30 to SEQ ID NO 32 and SEQ ID NO 34 to SEQ ID NO 36,
SEQ ID NO 38 to SEQ ID NO 40 and SEQ ID NO 42 to SEQ ID NO 44,
SEQ ID NO 46 to SEQ ID NO 48 and SEQ ID NO 50 to SEQ ID NO 52,
SEQ ID NO 54 to SEQ ID NO 56 and SEQ ID NO 58 to SEQ ID NO 60,
SEQ ID NO 62 to SEQ ID NO 64 and SEQ ID NO 66 to SEQ ID NO 68,
SEQ ID NO 70 to SEQ ID NO 72 and SEQ ID NO 74 to SEQ ID NO 76,
SEQ ID NO 78 to SEQ ID NO 80 and SEQ ID NO 82 to SEQ ID NO 84,
SEQ ID NO 86 to SEQ ID NO 88 and SEQ ID NO 90 to SEQ ID NO 92,
SEQ ID NO 94 to SEQ ID NO 96 and SEQ ID NO 98 to SEQ ID NO 100,
SEQ ID NO 102 to SEQ ID NO 104 and SEQ ID NO 106 to SEQ ID NO 108,
SEQ ID NO 110 to SEQ ID NO 112 and SEQ ID NO 114 to SEQ ID NO 116, and/or
SEQ ID NO 118 to SEQ ID NO 120 and SEQ ID NO 122 to SEQ ID NO 124.

56. A set of annealing primers and probes usable in a process according to claim 43 for determining bacterial germ counts of HPC22-HPC37 and/or of Coliforms comprising one or more polynucleotides with a nucleotide sequence selected from SEQ ID NO 1 to SEQ ID NO 6 and/or SEQ ID NO 7 to SEQ ID NO 12.

57. A set of primers and probes according to claim 50, wherein the set comprises primers and probes with the nucleotide sequences of
SEQ ID NOs 1 to 12,
SEQ ID NOs 1 to 6, or
SEQ ID NOs 7 to 12, respectively.

58. A set of complementary templates according to claim 53, wherein the set comprises primers and probes with the nucleotide sequences of
SEQ ID NOs 14, 15, 16, 18, 19, 20, 22, 23, 24, 26, 27 and 28,
SEQ ID NO 1 to SEQ ID NO 6, or
SEQ ID NO 7 to SEQ ID NO 12, respectively.

59. A set of complementary templates for hybridizing annealing primers and probes usable in a process according to claim 43 for determining bacterial germ counts of Escherichia coli, Pseudomonas aeruginosa, Enterococcus faecalis, Clostridium perfringens, Salmonella enterica, Staphylococcus aureus, Campylobacter jejuni/coli, Listeria monocytogenes, Shigella flexneri, Methicillin resistant Staphylococcus aureus, Legionella pneumophila and/or of Mycobacterium tuberculosis comprising one or more polynucleotides with a nucleotide sequence selected from
SEQ ID NO 30 to SEQ ID NO 32 and SEQ ID NO 34 to SEQ ID NO 36,
SEQ ID NO 38 to SEQ ID NO 40 and SEQ ID NO 42 to SEQ ID NO 44,
SEQ ID NO 46 to SEQ ID NO 48 and SEQ ID NO 50 to SEQ ID NO 52,
SEQ ID NO 54 to SEQ ID NO 56 and SEQ ID NO 58 to SEQ ID NO 60,
SEQ ID NO 62 to SEQ ID NO 64 and SEQ ID NO 66 to SEQ ID NO 68,
SEQ ID NO 70 to SEQ ID NO 72 and SEQ ID NO 74 to SEQ ID NO 76,
SEQ ID NO 78 to SEQ ID NO 80 and SEQ ID NO 82 to SEQ ID NO 84,
SEQ ID NO 86 to SEQ ID NO 88 and SEQ ID NO 90 to SEQ ID NO 92,
SEQ ID NO 94 to SEQ ID NO 96 and SEQ ID NO 98 to SEQ ID NO 100,
SEQ ID NO 102 to SEQ ID NO 104 and SEQ ID NO 106 to SEQ ID NO 108,
SEQ ID NO 110 to SEQ ID NO 112 and SEQ ID NO 114 to SEQ ID NO 116, and/or
SEQ ID NO 118 to SEQ ID NO 120 and SEQ ID NO 122 to SEQ ID NO 124.
